SEQUENCE LISTING (4 sequences)

SEQ ID NO: 1 - protein, 795 aa, Mus musculus
MSGVSEEDPEGLAPQGLPALGGACLATMDKKLNVLTEKVDRLLHFQEDVTEKLQCVCQGMDHLEQDLHRLEASRELSLAGSGSTPPTTAQAAWPEVLELVRAVRQEGAQHGARLEALFKMVVAVDRAITLVGSTFQNSKVADFIMQGTVPGRKGSLADGPEENKEQAEVAGVKPNHVLTTGGVQADASRTLWEESQKEDIPVRTVEGLPLIINTSLKGADLTQAGASLRQGVEVLGPGQVPLPTEAESRLPETASENTGATLELSVAIDRISEVLTSLKMSQGGGQETSSSKPDCWLSEEAMRLSSGPLPQPLGPLTPDSDIHSGDALPRIPINMQEMATPGELLETQSGSPIGSAEAPGLGTVLEDQIPKGARPFPPLPKRSSNNGGMSAEEEIGSGAEPMRGPSLATRDWRDETVGTTDLQQGIDPGAVSPEPGKDHAAQGPGRTEAGRLSSAAEAAIVVLDDSAAPPAPFEHRVVSIKDTLISAGYTVSQHEVLGGGRFGQVHRCTERSTGLALAAKIIKVKNVKDREDVKNEVNIMNQLSHVNLIQLYDAFESKSSFTLIMEYVDGGELFDRITDEKYHLTELDVVLFTRQICEGVHYLHQHYILHLDLKPENILCVSQTGHQIKIIDFGLARRYKPREKLKVNFGTPEFLAPEVVNYEFVSFPTDMWSVGVITYMLLSGLSPFLGETDAETMNFIVNCSWDFDADTFKGLSEEAKDFVSRLLVKEKSCRMSATQCLKHEWLSHLPAKASGSNVRLRSQQLLQKYMAQSKWKKHFHVVTAVNRLRKFPTCP

SEQ ID NO: 2 - protein, 795 aa, Homo sapiens
MDTKLNMLNEKVDQLLHFQEDVTEKLQSMCRDMGHLERGLHRLEASRAPGPGGADGVPHIDTQAGWPEVLELVRAMQQDAAQHGARLEALFRMVAAVDRAIALVGATFQKSKVADFLMQGRVPWRRGSPGDSPEENKERVEEEGGKPKHVLSTSGLQSDAREPGEESQKADVLERTAERLPPIRASGLGADPAQAVVSPGQGDGVPGPAQAFPGHLPLPTKVEAKAPETPSENLRTGLELAPAPGRVNVVSPSLEVAPGAGQGASSSRPDPEPLEEGTRLTPGPGPQCPGPPGLPAQARATHSGGETPPRISIHIQEMDTPGEMLMTGRGSLGPTLTTEAPAAAQPGKQGPPGTGRCLQAPGTEPGEQTPEGARELSPLQESSSPGGVKAEEEQRAGAEPGTRPSLARSDDNDHEVGALGLQQGKSPGAGNPEPEQDCAARAPVRAEAVRRMPPGAEAGSVVLDDSPAPPAPFEHRVVSVKETSISAGYEVCQHEVLGGGRFGQVHRCTEKSTGLPLAAKIIKVKSAKDREDVKNEINIMNQLSHVNLIQLYDAFESKHSCTLVMEYVDGGELFDRITDEKYHLTELDVVLFTRQICEGVHYLHQHYILHLDLKPENILCVNQTGHQIKIIDFGLARRYKPREKLKVNFGTPEFLAPEVVNYEFVSFPTDMWSVGVITYMLLSGLSPFLGETDAETMNFIVNCSWDFDADTFEGLSEEAKDFVSRLLVKEKSCRMSATQCLKHEWLNNLPAKASRSKTRLKSQLLLQKYIAQRKWKKHFYVVTAANRLRKFPTSP

SEQ ID NO: 3 - DNA, 3071 bp, Mus musculus
gagtcaggagggcaggctgaggctctgcagcaaggctcttaccagcggaggacaatggccttccagatgttagcaccaaagtgaaaggcagatccgcagatccgtggtgctgcaggttccttgcctttgtccttagcaaacccgaaatgggatgtcaggagtttcagaggaggacccagagggtctggccccccagggtctgccagcattgggcggagcctgcttagccaccatggacaaaaaacttaatgtgctgactgagaaggtcgacagactcttgcatttccaagaagatgtcacagagaagctgcagtgtgtgtgccaaggcatggatcacctggaacaagatctgcatcggctggaggcctcccgggagttgagtctggcagggtctggcagcactcccccaaccacggctcaggccgcatggcctgaggtcctggagctggtgagggctgtgcggcaggagggtgcccagcatggtgccaggctcgaagccctcttcaagatggtggtggctgtggacagggctattactttggtagggtccacattccagaattccaaggtggctgatttcatcatgcaaggaaccgtgcccgggagaaaaggcagtctggccgatggcccggaggagaacaaggagcaagcagaagttgctggagtgaagccaaaccatgtactgactacaggaggtgtgcaagctgacgcctctaggacgctgtgggaagagagccaaaaggaggacatacctgtgcgaacagtggaggggctgcctctcatcatcaatacgtcactgaagggagctgacctaacccaggcaggagcctcactgaggcagggagttgaagttcttggcccaggccaagtacccctacccacagaagcagaatccaggcttcctgagacagccagtgagaacactggagccaccctggaattgtccgtagcaatcgacagaatcagcgaggtcctcactagcctcaagatgtcacaaggtggtggtcaagaaacctcatccagcaagcctgactgttggctttcagaagaggccatgaggctgagttcagggcctctccctcagcccctaggaccactaactcctgacagtgacattcacagtggtgatgcacttcccaggatccctatcaatatgcaagagatggctactcctggggagttgcttgagacccaaagtggcagtcccattggctctgcagaagctccaggccttgggactgtgttagaagaccagatccctaaaggagccagaccatttccacccctgccaaagaggagcagcaacaatggtggcatgagtgcagaggaggagatagggtctggggctgagcctatgagaggaccaagcttggctacaagggactggagagatgagactgttgggaccacagacctgcagcaaggcatagacccaggagcagtgagccctgagcctgggaaggaccacgcagcccagggcccaggaagaactgaagctggaaggctatcttctgctgcagaggctgccattgtggttctagatgacagcgcagcacccccagccccttttgaacaccgggtagtgagcatcaaagataccctgatctcagcaggctacacggtatcccaacatgaagtcttaggagggggtcggtttggccaggtgcacaggtgtacagagaggtctacaggccttgcactggcagccaagatcatcaaagtgaagaacgtaaaggaccgggaggatgtgaagaatgaggtcaacatcatgaaccagctcagccacgtaaacttgatccaactttatgatgcgtttgagagcaagagcagcttcactctgatcatggagtatgtggatggaggcgaactctttgaccggatcacggatgagaagtaccacctcactgagttggatgtggtcttgttcacgaggcagatctgtgagggtgtgcattacctgcatcagcactatatcctgcacctggacctcaagcctgagaacatattgtgtgtcagccagacagggcatcaaattaagatcattgactttgggctggctagaagatacaagcctcgggagaagctaaaggtgaactttggtactccggagttcctggccccagaagttgttaactatgagtttgtgtcatttccaacagacatgtggagtgtgggagttatcacctacatgctactcagtggtttgtccccatttctaggggagacagatgcagagaccatgaattttattgtgaactgcagctgggatttcgatgctgataccttcaaagggctgtcggaggaagccaaggactttgtttcccggttactggtcaaagagaagagctgtaggatgagcgccacacagtgcctgaaacacgagtggttaagtcacctgcctgccaaagcctcgggctccaacgttcgcctcagatcccaacaactgctgcagaaatatatggctcagagtaaatggaagaaacatttccacgtggtgactgcagtcaacaggctacggaaatttccaacgtgtccctaatcttcaactctgctgttccactgggcctgggaattcttgaggcaacacgaagtggtaatatgaagagattactcaagattttacggagattggcgctttgctattattgatttttcttattttgcaaagaatgatggaaggaagcaagaaagaaagaaaagaagaaaagggggaagaaaaggaaaaggcagaaagcaaggaaacaggctacgttgttgctcttcttgtaggtgaaagtgtttttattaaaagccctaggaatgtttttctgcctcgtaaggtcagcaggtctcatatgctgcttgctcccccgcacccttccttttggtaataagagcaggcacgctcaggatgggcagggaaatcctacttggcttttggtcaaatttgaattctaaacttgtcatgattaaagaagccagtagggagtgaggtatggaagagggaggaattaggtccaacagtgggggatgaatttgacggaaacattgtataaaattcttaaagaattaataaaatatatttttaaagg

SEQ ID NO: 4 - DNA, 2760 bp, Homo sapiens
aggagtctgtcagctacggaggacaatgaccttgcagacaccaccgcctgagtgagaaccaggggtctgtgcctctcctcattccccgctcttgcccttgtcaagcctgcaccagcatgtcaggaacctccaaggagagtctggggcatggggggctgccagggttgggcaagacctgcttaacaaccatggacacaaagctgaacatgctgaacgagaaggtggaccagctcctgcacttccaagaagatgtcacagagaagttgcagagcatgtgccgagacatgggccacctggagcggggcctgcacaggctggaggcctcccgggcaccgggcccgggcggggctgatggggttccccacattgacacccaggctgggtggcccgaggtcctggagctggtgagggccatgcagcaggatgcggcccagcacggtgccaggctggaggccctcttcaggatggtggctgcggtggacagggccatcgctttggtgggggccacgttccagaaatcaaaggtggcggatttcctcatgcaggggcgtgtgccctggaggagaggcagcccaggtgacagccctgaggagaataaagagcgagtggaagaagagggaggaaaaccaaagcatgtgctgagcaccagtgggttgcagtctgatgccagggagcctggggaagagagccagaaggcggacgtgctggaaaggacagcggagaggctgccccccatcagagcgtcagggctgggagctgaccccgcccaggcagtggtctcaccgggccagggagatggtgttcctggcccagcccaggcattccctggccacctgcccctgcccacaaaggtggaagccaaggctcctgagacacccagcgagaacctcaggactggcctggaattggctccagcacccggcagggtcaatgtggtctccccgagcctggaggttgcaccaggtgcaggacaaggagcatcgtccagcaggcctgaccctgagcccttagaggaaggcacgaggctgactccagggcctggccctcagtgcccagggcctccagggctgccagcccaggccagggcaacccacagtggtggagaaacacctccaaggatctccatccacatacaagagatggatactcctggggagatgctgatgacaggcaggggcagccttggacccaccctcaccacagaggctccagcagctgcccagccaggcaagcagggcccacctgggaccgggcgctgcctccaagcccctgggactgagcccggagaacagacccctgaaggagccagagagctctccccgctgcaggagagcagcagccccgggggagtgaaggcagaggaggagcaaagggctggggccgagcctggcacgagaccaagcttggccaggagtgacgacaatgaccacgaggttggggccctgggcctgcagcagggcaaaagcccaggggcgggaaaccctgagcctgagcaggactgtgcagccagggctccggtgagagctgaagcagtaaggaggatgcccccaggcgccgaggctggcagcgtggttctggatgacagtccggccccaccagctccttttgaacaccgggtagtgagcgtcaaggagacctccatctctgcgggttacgaggtgtgccagcacgaagtcttgggagggggtcggtttggccaggtccacaggtgcacagagaagtccacaggcctcccactggctgccaagatcatcaaagtgaagagcgccaaggaccgggaggacgtgaagaacgagatcaacatcatgaaccagctcagccacgtgaacctgatccagctctatgacgccttcgagagcaagcacagctgcacccttgtcatggagtacgtggacgggggtgagctcttcgaccggatcacagatgagaagtaccacctgactgagctggatgtggtcctgttcaccaggcagatctgtgagggtgtgcattacctgcaccagcactacatcctgcacctggacctcaagccggagaacatattgtgcgtcaatcagacaggacatcaaattaagatcattgactttgggctggccagaaggtacaagcctcgagagaagctgaaggtgaacttcggcactcctgagttcctggccccagaagtcgtcaattatgagtttgtctcattccccacagacatgtggagtgtgggagtcatcacctacatgctactcagtggcttgtccccatttctaggggaaacagatgcagagaccatgaatttcattgtaaactgtagctgggattttgatgctgacacctttgaagggctctcggaggaggccaaggactttgtttcccggttgctggtcaaagagaagagctgcagaatgagtgccacacagtgcctgaaacacgagtggctgaataatttgcctgccaaagcttcaagatccaaaactcgtctcaaatcccaactactgctgcagaaatacatagctcaaagaaaatggaagaaacatttctatgtggtgactgctgccaacaggttaaggaaatttccaacttctccctaatcttcaactctgctgctccaatgggtccagaaattactgaggccagtggtgaagtgaagagatgactcaaacatttaaataatttggcttttggtattattgattccacttatttgtaaatggttatggctgctgcttcctgtggatgaaagtggctgtaagagctcctagacgtttctgctgt

(19) United States
(12) Patent Application Publication
(10) Pub. No.: US 2010/0129815 A1
(43) Pub. Date: May 27, 2010

(54) IDENTIFICATION OF CARDIAC SPECIFIC MYOSIN LIGHT CHAIN KINASE

(75) Inventor: Hideko Kasahara, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: UNIVERISTY OF FLORIDA RESEARCH FOUNDATION INC., GAINESVILLE, FL (US)

(21) Appl. No.: 12/595,025

(22) PCT Filed: Apr. 14, 2008
(86) PCT No.: PCT/US08/60201

§ 371 Date: Oct. 7, 2009

Related U.S. Application Data
(60) Provisional application No. 60/911,770, filed on Apr. 13, 2007.

(51) Int. Cl. C12Q 1/68; C12Q 1/48

(52) U.S. Cl. 435/6; 435/15

(57) ABSTRACT

The subject invention pertains to the association of a MLCK homologue with cardiac tissue and methods of identifying candidate compounds that can modulate the activity of this MLCK homologue and the expression of MLCK gene product. Additionally, methods of treating heart failure are also provided by the subject invention.

### CROSS-REFERENCE TO RELATED APPLICATION

This application claims the benefit of U.S. Provisional Application Ser. No. 60/911,770, filed Apr. 13, 2007, the disclosure of which is hereby incorporated by reference in its entirety, including all figures, tables and amino acid or nucleic acid sequences.

This invention was made with government support under NHLBI, National Institutes of Health, contract number IR01 HL081577. The government has certain rights in the invention.

### BACKGROUND OF THE INVENTION

Muscle contraction depends on the ATP-driven sliding of highly organized arrays of actin filaments against arrays of myosin II filaments. Phosphorylation of both myosin heavy- and light-chains affects both motor activity and thick filament assembly (1).

In nonmuscle cells, myosin II probably exists in two different conformational states: a bent state (inactive state, 10S) in which the tail domain apparently interacts with the motor head and an extended state (active state, 6S) that is capable of forming bipolar filaments and exposing its actin-binding site. Phosphorylation of regulatory myosin light chain (MLC) by Ca²⁺/calmodulin-dependent myosin light chain kinase (MLCK) likely causes the myosin II to preferentially assume the extended state, which promotes its assembly into a bipolar filament leading to cell contraction (1). In smooth muscle cells, phosphorylation of MLC2 by smooth muscle-MLCK is thought to be responsible for the initiation of contraction (2). In skeletal and cardiac muscles, however, initiation of muscle contraction depends on voltage-gated L-type Ca²⁺ channels in the plasma membrane and T-tubules. Increased local Ca²⁺ concentrations allows the sarcoplasmic reticulum to release large amounts of Ca²⁺, which binds to troponin C followed by myosin-actin cross-bridge formation. During this process, MLCK potentiates peak tension in skeletal muscle (1,3), and the force and the rate of cross-bridge recruitment in cardiac myocytes (4,5).

To date, skeletal- and smooth muscle-MLCKs have been characterized (3). Mouse skeletal muscle-MLCK gene (mylk1) is located on chromosome 2, encodes 613 amino acids and is predominantly expressed in skeletal muscle, while mouse smooth muscle-MLCK gene (mylk2) is located on chromosome 16, encodes three different isoforms by alternative promoters, the long form has 1950 amino acids and the short form has 1031 amino acids, and they are expressed in several tissues (6,7). The shortest form lacking a catalytic domain, called telokin has also been characterized (3). Generally, MLCKs have preference for their MLC substrate isolated from the same tissue. For instance, the smooth muscle MLCK phosphorylates smooth muscle MLC2, but it does not preferentially phosphorylate skeletal MLC2 and cardiac MLC2v. The amino acid sequence of substrates appears to be critical for catalytic activity of smooth muscle MLCK, particularly an arginine residue located at the third position amino-terminal of the phosphorylated serine residue, which is present in smooth muscle MLC (Arg-Ala-Thr-Ser), but absent in skeletal MLC (Gly-Gly-Ser-Ser) as well as MLC2v (Gly-Gly-Thr-Ser) (8-10).

On the other hand, skeletal MLCK has little substrate specificity, which phosphorylates skeletal-, smooth muscle and cardiac MLCs (11,12). In addition, mutations in human skeletal MLCK on human chromosome 20 were mapped to a disease locus for familial cardiac hypertrophy (OMIM 606566), suggesting that the abnormal function of skeletal MLCK stimulates cardiac hypertrophy (13). However, the abundance of skeletal MLCK expression in the heart is controversial (13-15). Short-form (130 kDa) smooth-muscle MLCK is expressed in the heart at lower levels than those detected in smooth muscle-rich organs such as gut, uterus and lung (6,7). Gene targeted mice for skeletal MLCK as well as for smooth muscle MLCK appear to have normal cardiac function (15,16). These results suggest that an additional MLCK that is preferentially expressed in the heart is plausible because MLC2 phosphorylation in cardiac muscle is a key regulator of heart contraction (5). Indeed, several cardiac proteins contain MLCK-like domains including titin-kinase and obscurin-MLCK kinase. Although a substrate for obscurin-MLCK kinase remains to be identified, the substrate for titin-kinase is not MLC, but Z-disc localizing protein telethonin (titin-cap protein) (17).

In the process of identifying genes regulated by the cardiac homeobox transcription factor, NRx2-5, a gene product highly homologous to the previously characterized skeletal and smooth muscle MLCK was identified; however, this gene product is distinctly different in its amino-terminal domain. The sequence of this MLCK homologue has been available (NCBI UniGene No. Rn. 43838-rat, Mm32804-mouse (each of which is hereby incorporated by reference in its entirety), yet its tissue-specific expression pattern and function have not been studied. Limited information regarding this MLCK gene has sometimes confounded its identity with the previously characterized skeletal and smooth muscle MLCKs. In this application, the initial characterization of the MLCK homologue, including its cardiac-specific expression, intracellular localization, catalytic activity and potential functions in sarcomere organization and cardiac contraction are described and uses for the homologue are discussed.

### BRIEF SUMMARY OF THE INVENTION

The subject invention pertains to the association of a MLCK homologue with cardiac tissue and methods of identifying candidate compounds that can modulate the activity of this MLCK homologue and the expression of MLCK gene product. Additionally, methods of treating heart failure are also provided by the subject invention.

### BRIEF DESCRIPTION OF THE DRAWINGS

FIGS. 1A-1B. Identification of cardiac-specific MLCK as an NRx2-5downstream target gene. (A) Northern blotting showed that NRx2-5 knockdown using adenovirus shRNA (1A-a) reduced expression of cardiac MLCK 48 hr after adenovirus infection (a, lane 1 vs. 2). Further reduction of cardiac MLCK expression was detected 96 hr after adenovirus infection (a, lane 3 vs. 4). Tamoxifen-induced NRx2-5 knockout demonstrates reduction of cardiac MLCK expression at postnatal day 2 and further reduction at postnatal day 4 (1A-b). Of note, tamoxifen was injected to the pregnant female within 24 hr before delivery. Coni, control RNAi; Nkxi, NRx2-5-RNAi; fl, floxed-NRx2-5; fl/fl, homozygous for floxed-NRx2-5; w, wild-type; w/w, homozygous for wild-type; cre, cre-transgenic; D2, postnatal day 2; D4, postnatal day 2. (B) Tissue specific expression of cardiac MLCK mRNA was examined by Northern blotting (top panel) and compared to that of skeletal MLCK (middle panel) in the neonatal stage (1B-a) and adult stage (1B-b). Cardiac MLCK is expressed in both ventricle and atrium with similar expression levels. Increased loading of RNA isolated from adult atrium resulted in higher expression of MLCK in adult atrium vs. ventricle (*, lane 4, panel b). Br, brain; Lu, lung; V, ventricle; A, atrium; To, tong; M(l), leg muscle; M(b), back muscle; Li, liver; Ki, kidney; St, stomach; In, intestine.

FIGS. 2A-2C. Structure of mouse cardiac MLCK protein compared to skeletal and smooth muscle MLCKs. (A) Diagonal dot matrix comparison between cardiac and skeletal MLCK protein. Homology between the two was observed only at the carboxyl-terminus. (B) Amino acid sequence of cardiac MLCK with alignment among cardiac, skeletal and smooth muscle (long form) MLCK at the carboxyl-terminus. Identical amino acids among three proteins are shaded. Ca²⁺/calmodulin binding kinase regulatory domain locating carboxyl-terminus to catalytic domain; two contiguous serine residues, which are targets of upstream kinases (3) and two additional autophosphorylation sites (45) identified in the smooth muscle MLCK are indicated in green. (C) Schematic structure of cardiac MLCK protein compared with skeletal and three forms of smooth muscle MLCK. Overall homology of the kinase domain and regulatory domain between cardiac and skeletal MLCK is 58%, and cardiac and smooth muscle MLCK is 44%. Chromosome localization of three MLCKs in mouse and human is shown. Protein sequence are cited from mouse skeletal MLCK (XP_{—}979674), smooth muscle MLCK (long) NP647461, smooth muscle MLCK (short) cited from (6), telokin (AAG34169).

FIGS. 3A-3E. Cardiac MLCK protein expression and its intracellular localization. (A) Western blotting using anti-cardiac MLCK antibody against GST-cardiac MLCK(28-463 aa) (lanes 1, 2) detected HA-tagged full length cardiac-MLCK protein (lane 3), but not HA-tagged full length skeletal MLCK (lanes 4, 5). One ng of GST-MLCK is close to 0.63 μl of purified HA-tagged full length cardiac-MLCK protein (lane 3). Anti-HA antibody recognizes HA-tagged cardiac and skeletal MLCK (lanes 3-5). (B) Western blotting using anti-cardiac MLCK antibody detected similar amount of MLCK protein in protein lysates isolated from mouse neonatal ventricles (lane 1) and atria (lane 2). (C) Cardiac MLCK protein expression in the heart lysates is equivalent to 2.3 ng of GST-cardiac MLCK in 5 μg of heart lysates (estimated concentration in our experimental condition 0.45 μg/mg). The antibody against the amino-terminus domain of cardiac MLCK does not appear to cross-react with other proteins using similar amounts of tissue lysates from skeletal muscle and lung in which skeletal or smooth muscle MLCK is abundantly expressed (6). (D) HA-tagged MLCK proteins overexpressed by adenovirus were co-immunostained with anti-MLCK and anti-HA antibody. Merged image is also shown. (E) Co-immunostaining of cardiac MLCK, actin (phalloidin) and MLC2v. Endogenous cardiac MLCK protein is localized diffusely in the cytoplasm with occasional formation of a striated pattern (arrows), which overlaps with actin but not with striated MLC2v. Arrowheads indicate non-cardiomyocyte weakly detected by phalloidin, but not by cardiac MLCK, nor MLCK2v antibodies. Ad-HA-MLCK, adenovirus encoding HA-tagged cardiac MLCK; V, ventricle; A, atrium. Bars 10 μM.

FIGS. 4A-4D. Cardiac MLCK phosphorylates MLC2 and potentially MLCK itself. (A) In vitro cardiac MLCK phosphorylation of MLC2v and 2a. HA-tagged cardiac MLCK expressed in 293 cells were immunoprecipitated with anti-HA antibody, then mixed with GST (lane 1), GST-MLC2v (lane 2), or GST-MLC2a (lane 3) in the presence of [³²P]-'γATP. Control experiments in the absence of anti-HA antibody were shown in lanes 4-6. (4B-a) Autoradiogram of MLC2v phosphorylation at various concentrations (0.012-3 μM) catalyzed by cardiac MLCK (top panel) and skeletal MLCK (bottom panel) in the presence of EGTA or Ca²⁺/calmodulin. (4B-b) Calculated kinetic values of cardiac MLCK from Lineweaver-Burk plot showed Km 4.3±1.5 μM (without EGTA or Ca²⁺/calmodulin), 2.9±0.8 (with EGTA) and 3.9±1.2 (with Ca²⁺/calmodulin); Vmax 0.26±0.06 μmol/min/mg (without EGTA or Ca²⁺/calmodulin), 0.18±0.03 (with EGTA) and 0.18±0.03 (with Ca²⁺/calmodulin). Values are expressed as mean±S.E, n=2. (C) MLC2 phosphorylation was examined in neonatal cardiomyocytes with increased expression of cardiac MLCK (panel a, lanes 2, 3 vs. lane 1), or decreased level of cardiac MLCK using three different shRNAs (panel b, lanes 2, 3 vs. lane 1). The relative amounts of phosphorylated MLC2v to total MLC2v protein are shown below in comparison to control cardiomyocytes. The relative values are expressed as mean±S.E, n=2 for Ad-MLCK, n=4 for RNAi-MLCK. Reduced expression of cardiac MLCK by three different shRNA is detected by Western blotting with anti-MLCK antibody (panel c, lane 1, shRNA with scrambled sequence; lanes 2-4, shRNA targeting to different sequences). (4D-a) Cardiac MLCK immunoprecipitated from neonatal ventricular cardiomyocytes were blotted with anti-phospho-serine or phospho-tyrosine antibody. Phospho-serine antibody reacted to cardiac MLCK proteins. (4D-b) In vitro kinase assay showed ³²P incorporated HA-tagged MLCK expressed in 293 cells.

FIGS. 5A-5B. Effects of cardiac MLCK in sarcomere organization and cell shape. (A) Overexpression of cardiac MLCK by adenovirus promotes sarcomere organization detected by phalloidin compared to those infected with control adenovirus encoding beta-galactosidase (a vs. b). (B) Reduced expression of cardiac MLCK using two different shRNAs (RNAi-1 and 2) does not appear to disturb sarcomere organization with only slight changes in peripheral structure (arrows) (a, b, vs. c, d). Bars 10 μM.

FIGS. 6A-6D. Cardiac MLCK potentiates cardiomyocyte contraction. (A) Representative tracings of cell motion transients (top) and simultaneous Ca²⁰⁺ transient (bottom) in an isolated rat neonatal cardiomyocytes. Fifty moi of adenovirus encoding beta-galatosidase (a, c) or cardiac-MLCK (b, d) were infected. Continuous recording for 30 seconds (a, b) and a single contraction (c, d) are shown. (B) Summarized data obtained from multiple cardiomyocytes demonstrate that increased cardiac MLCK expression significantly increases cell motion, +dL/dT (contraction) and −dL/dT (relaxation) without a significant change in Ca²⁺ amplitude and decay. (C) Representative tracings of a cell motion in a cardiomyocyte infected with adenovirus-shRNA with scrambled sequence (RNAi-control, a) or targeting to cardiac MLCK (RNAi-MLCK, b). Two different Ca²⁺ concentrations in the superfusate (1.2 mM and 2.5 mM) are tested. Higher Ca²⁺ concentration potentiates cardiac contraction in control cardiomyocytes, but not in MLCK knockdown cardiomyocytes. (D) Summarized data of the percent changes between 1.2 mM and 2.5 mM Ca²⁺ concentrations measured in individual cardiomyocytes.

FIG. 7. Cardiac MLCK expression in post-myocardial infarction heart failure mouse model. Non-infarcted upper septum dissected from three sham-operated (lanes 1, 2), two moderate (lanes 4, 5), and two severe (lanes 6, 7) heart failure mice were analyzed for cardiac MLCK protein expression. Decreased cardiac MLCK expression was detected in mice with severe heart failure compared with sham-operated mice. Values in sham-operated (n=2), moderate (n=2), and two severe (n=2) heart failure mice are as follows: heart weight/body weight ratio (mean±SD), 4.23±0.74 vs. 5.41±0.35 vs. 9.52±1.87 mg/g, respectively; heart weight/tibial length (mean±SD), 5.87±0.49 vs. 7.97±1.32 vs. 15.0±1.91 mg/mm, respectively. Experimental conditions have been described previously (26).

FIGS. 8A-8I. Cardiac MLCK expression and MLC2v phosphorylation in mice with NRx2-5 knockout, aging and post-myocardial infarction heart failure. (A, B) Decreased expression of cardiac MLCK mRNA and protein in NRx2-5 knockout hearts at postnatal day 12. Skeletal MLCK mRNA was not detected by Northern blotting. (C) Unphosphorylated (left, with higher pI) and phosphorylated (right, with lower pI) MLC2v examined in two-dimensional electrophoresis followed by Western blotting with anti-MLC antibody. Relative amounts of phosphorylated to total MLC2v are shown (mean±S.E., n=2). (D) Expression of cardiac MLCK and NRx2-5 mRNA in neonatal, adult (4 month) and aged hearts (18 month). Relative expression of cardiac MLCK normalized to GAPDH is shown with the value in neonatal heart defined as 1. (E) Expression of cardiac MLCK protein in embryonic day 10.5, neonatal, adult and aged hearts. Relative expression of cardiac MLCK normalized to GAPDH is shown with the value in neonatal heart defined as 1. (F) Level of MLC2v phosphorylation in young (PD 12) and aged hearts. Relative amounts of phosphorylated to total MLC2v are shown (mean±S.E.; young, n=3; old, n=6 from two mice at 18 and 21 month). (G) Non-infarcted upper septal tissue dissected from mice 3 weeks after coronary ligation (3 month old) was analyzed for cardiac MLCK mRNA expression: two sham-operated (lanes 1, 2) and two heart failure mice (lanes 3, 4). Values of heart weight/body weight are indicated. Additional experimental conditions and parameters of cardiac function are described previously³⁵ and in Methods. Relative expression of cardiac MLCK normalized to GAPDH is shown with the value in sample 1 defined as 1. (H) Cardiac MLCK protein expression in tissue lysates from the same mice used in FIG. 7G is shown with the value in sample 1 defined as 1. (I) Level of MLC2v phosphorylation examined in mice with sham-operated and heart failure. Relative amounts of phosphorylated to total. MLC2v are shown (mean±S.E.; sham, n=4 from 2 mice; heart failure, n=4 from 2 mice). MI, myocardial infarction.

FIG. 9. Smooth muscle MLCK expression in the heart, skeletal muscle and lung. Anti-smooth muscle MLCK monoclonal antibody clone K36 recognized GST-smooth muscle MLCK(aa 1-337 of short form MLCK) (lanes 1-3), but not two other GST-fused carboxyl-termini of smooth muscle MLCK(aa 274-860 and 829-1031) (lanes 4, 5). Expression of 130-kDa short-form smooth muscle MLCK protein is detected in the heart (estimated concentration 0.025-0.05 μg/mg), skeletal muscle, and more abundantly in the lung (estimated concentration 0.5-1 μg/mg) (lanes 6-8, arrowhead). Additional proteins with approximate MW 60 kDa are also recognized (* in lanes 6-8).

### BRIEF DESCRIPTION OF THE SEQUENCES

SEQ ID NO: 1 is the polypeptide sequence of murine cardiac MLCK.

SEQ ID NO: 2 is the polypeptide sequence of human MLCK.

SEQ ID NO: 3 is the polynucleotide sequence encoding murine cardiac MLCK.

SEQ ID NO: 4 is the polynucleotide sequence encoding human MLCK.

### DETAILED DISCLOSURE OF THE INVENTION

Host cells transformed by a vector comprising a nucleic acid encoding the MLCK polypeptide can be used for a variety of purposes, including the screening of candidate compounds for the ability to modulate MLCK expression or activity. In certain aspects of the invention, it is preferable to utilize eukaryotic cells, such as MLCK deficient or defective cardiomyocytes, as host cells for the expression of MLCK polypeptide. Exemplary nucleic acids in this aspect of the invention are SEQ ID NO: 3 (see also NCBI Accession No. NM_{—}175441, which is hereby incorporated by reference in its entirety) or SEQ ID NO: 4 (see also NCBI Accession No. NM_{—}182493, which is hereby incorporated by reference in its entirety).

The term “modulate(s)” refers to the ability of a candidate compound to upregulate/increase or downregulate/decrease MLCK gene expression or MLCK polypeptide activity.

The term “biological sample” is used to refer to cardiomyocytes and/or cardiac fibers that are obtained from an individual or grown in culture from cell lines.

The terms “subject(s)” or “individual(s)” may be used interchangeably throughout this specification and include human and non-human animals.

The term “MLCK polypeptide” includes the murine and human form of cardiac specific MLCK polypeptides. The murine MLCK is identified in SEQ ID NO: 1 (see also NCBI Accession No. NP_{—}780650, which is hereby incorporated by reference in its entirety). The human form of the MLCK polypeptide is identified in SEQ ID NO: 2 (see also NCBI Accession No. NP_{—}872299, which is hereby incorporated by reference in its entirety). The term “MLCK polypeptide” can also include catalytically active fragments of a MLCK polypeptide (e.g., a polypeptide that retains kinase activity or the ability to phosphorylate substrate).

The term “cardiac sample” is used to identify biological samples obtained from a test or control subject that are to be tested for MLCK expression. A “cardiac sample” is any cardiac tissue, including individual cardiomyocytes, populations of cardiomyocytes and cardiac fibers obtained from a subject.

The phrases “MLCK gene product” or “MLCK expression” refers to nucleic acids (e.g., mRNA or cDNA) obtained from the expression of the MLCK gene in a subject or individual or a host cell as well as to polypeptides and fragments thereof arising from the translation of mRNA into protein (i.e., MLCK polypeptide). In certain aspects of the invention, methods of detecting an increase or decrease in mRNA levels or polypeptide levels in response to candidate compounds are preferred.

### Methods of Identifying Subjects at Risk of Heart Failure:

The subject invention provides for methods of identifying individuals or subjects at risk of developing heart failure. In this aspect of the invention, individuals at risk of developing heart failure are identified as those individuals having lower levels of MLCK polypeptide as compared to normal or control individuals. Methods of identifying such individuals are based upon Western blot formats or standard immunoassays known to the skilled artisan and utilize MLCK polypeptide derived from cardiac or heart tissue (e.g., cardiomyocytes). For example, antibody-based assays such as enzyme linked immunosorbent assays (ELISAs), radioimmunoassays (RIAs), lateral flow assays, reversible flow chromatographic binding assay (see, for example, U.S. Pat. No. 5,726,010, which is hereby incorporated by reference in its entirety), immunochromatographic strip assays, automated flow assays, and assays utilizing peptide-containing biosensors may be employed for the detection of MLCK polypeptides (or fragments thereof) in biological samples obtained from an individual or subject. Assays and methods for conducting the assays are well-known in the art and the methods may test biological samples (e.g., cardiac tissue samples) qualitatively (presence or absence of MLCK polypeptide) or quantitatively (comparison of a biological sample from a subject against a sample obtained from normal (control) subjects. These assays can detect the full length MLCK polypeptide or fragments of the MLCK polypeptide that are recognized/specifically bound by MLCK specific antibodies.

The antibody-based assays can be considered to be of four types: direct binding assays, sandwich assays, competition assays, and displacement assays. In a direct binding assay, either the antibody or antigen is labeled, and there is a means of measuring the number of complexes formed. In a sandwich assay, the formation of a complex of at least three components (e.g., antibody-antigen-antibody) is measured. In a competition assay, labeled antigen and unlabelled antigen compete for binding to the antibody, and either the bound or the free component is measured. In a displacement assay, the labeled antigen is pre-bound to the antibody, and a change in signal is measured as the unlabelled antigen displaces the bound, labeled antigen from the receptor.

Lateral flow assays can be conducted according to the teachings of U.S. Pat. No. 5,712,170 and the references cited therein. U.S. Pat. No. 5,712,170 and the references cited therein are hereby incorporated by reference in their entireties. Displacement assays and flow immunosensors useful for carrying out displacement assays are described in: Kusterbeck et al., (1990); Kusterbeck et al., (1990a); Ligler et al., (1992); Ogert et al., (1992), all of which are incorporated herein by reference in their entireties. Displacement assays and flow immunosensors are also described in U.S. Pat. No. 5,183,740, which is also incorporated herein by reference in its entirety. The displacement immunoassay, unlike most of the competitive immunoassays used to detect small molecules, can generate a positive signal with increasing antigen concentration.

The term “antibody” includes monoclonal antibodies (including full length monoclonal antibodies), polyclonal antibodies, multispecific antibodies (e.g., bispecific antibodies), and antibody fragments so long as they exhibit the desired biological activity, particularly neutralizing activity. “Antibody fragments” comprise a portion of a full length antibody, generally the antigen binding or variable region thereof. Examples of antibody fragments include Fab, Fab', F(ab')₂, and Fv fragments; diabodies; linear antibodies; single-chain antibody molecules; and multi-specific antibodies formed from antibody fragments.

### Screening Assays:

The MLCK polypeptide can also be used in screening assays to identify candidate compounds suitable for the treatment of heart failure or myocardial infarction. In these assays, the MLCK polypeptide can be expressed in a host cell or cardiomyocytes containing the MLCK polypeptide can be used to for identifying candidate compounds that are able to modulate the activity of the MLCK polypeptide. For example, compound libraries can be screened of the ability to increase (agonize) or inhibit (antagonize) the level of expression of the MLCK gene or the activity of the MLCK polypeptide. Preferred compounds increase the expression of the naturally occurring MLCK gene or increase the activity of the MLCK polypeptide within cardiomyocytes or cardiac fibers.

Candidate compounds (e.g., agonists or antagonists) may be isolated from, for example, cells, cell-free preparations, chemical libraries or natural product mixtures. These agonists or antagonists may be natural or modified substrates, ligands, enzymes, receptors or structural or functional mimetics. Additional candidate compounds include small organic molecules, peptides, polypeptides and antibodies that bind to the MLCK polypeptide and increase or decrease (inhibit) its kinase activity.

Where the MLCK polypeptide is employed in screening techniques, the polypeptide can be provided free in solution or attached to a solid support. Alternatively, the MLCK polypeptide can be expressed within a cell, expressed at the cell surface or provided as a natural expression product in cardiomyocytes. In general, screening procedures involve contacting a MLCK polypeptide with a candidate compound to observe binding or the stimulation or inhibition of a biological activity. The stimulation or inhibition of a biological activity is compared to a control preparation that is not contacted with the candidate compound.

One biological activity that can be used to identify candidate compounds that modulate the activity of MLCK is kinase activity. For example, a kinase activity assay can be performed in the presence and absence of a candidate compound and the effect of the candidate compound on MLCK activity (e.g., the ability to phosphorylate a suitable substrate) is determined. Assays for MLCK kinase activity are well-known in the art; see, for example, U.S. Pat. No. 5,906,819. Alternatively, the ability of a candidate compound to modulate MLCK activity can be assessed by a series of kinase activity assays that are carried out in the presence of varying concentrations of the candidate compound (including a control assay containing no candidate compound). The extent of phosphate incorporation into substrate is determined for each assay in the series. Other assays for assessing phosphate incorporation into substrates are disclosed in published U.S. Application Number: 20040126860, which is hereby incorporated by reference in its entirety. A preferred method for identifying a candidate (an agonist or antagonist) compound of the MLCK polypeptide comprises:

(a) contacting a MLCK polypeptide with a candidate compound; and

(b) determining whether the candidate compound modulates MLCK activity.

In certain of the embodiments, simple binding assays may be used to detect the binding of a candidate compound to a MLCK polypeptide. Such binding can be detected by means of a label directly or indirectly associated with the candidate compound or in an assay involving competition with a labeled competitor. In another embodiment, competitive drug screening assays can be used to identify candidate compounds that interact with the active site of MLCK. For example, antibodies specific for the MLCK active site can be used to specifically compete with a candidate compound for binding to the MLCK active site.

Screening assays may include the detection of an increase or a decrease in the production of mRNA encoding the MLCK polypeptide or detection of increased or decreased levels of MLCK mRNA in cells (e.g., cardiomyocytes or cardiac fibers). For example, an ELISA assay that measures cell-associated levels of polypeptide using monoclonal or polyclonal antibodies by can be used to identify compounds that may inhibit or enhance the production of the MLCK polypeptide in transformed host cells, cardiomyocytes or cardiac fibers. The formation of binding complexes between the polypeptide and the compound being tested may then be measured. Alternatively, hybridization assays for detecting the level of MLCK mRNA can be performed according to methods well known in the art.

Another aspect of the invention provides methods of screening candidate compounds that comprise (a) detecting MLCK gene product signals from cells expressing the MLCK gene product; (b) contacting the cells with a candidate compound; (c) detecting gene product signals from cells contacted with the candidate compound; (d) comparing gene product signals from the cells from cells before and after contact with the candidate compound to determine the responsiveness of the MLCK gene to increased or decreased expression arising from contact with the candidate compound. The MLCK gene product can be either the MLCK polypeptide or mRNA expressed by the cell after contact with the candidate compound.

Methods for the detection/identification of increased or decreased expression of genes, such as the MLCK gene, exist and any suitable method for detection is encompassed by the instant invention. Typical assay formats utilizing nucleic acid hybridization includes, and are not limited to, 1) nuclear run-on assay, 2) slot blot assay, 3) northern blot assay (Alwine, et al., Proc. Natl. Acad. Sci. 74:5350), 4) magnetic particle separation, 5) nucleic acid or DNA chips, 6) reverse Northern blot assay, 7) dot blot assay, 8) in situ hybridization, 9) RNase protection assay (Melton, et al., Nuc. Acids Res. 12:7035 and as described in the 1998 catalog of Ambion, Inc., Austin, Tex.), 10) ligase chain reaction, 11) polymerase chain reaction (PCR), 12) reverse transcriptase (RT)-PCR (Berchtold, et al., Nuc. Acids. Res. 17:453), 13) differential display RT-PCR (DDRT-PCR), 14) nested PCR, 15) quantitative PCR or other suitable combinations of techniques and assays. Labels suitable for use in these detection methodologies include, and are not limited to 1) radioactive labels, 2) enzyme labels, 3) chemiluminescent labels, 4) fluorescent labels, 5) magnetic labels, or other suitable labels, including those set forth below. The general methods of PCR are well known in the art and are thus not described in detail herein. For a review of PCR methods, protocols, and principles in designing primers, see, e.g., Innis et al., PCR Protocols: A Guide to Methods and Applications, Academic Press, Inc. N.Y., 1990. PCR reagents and protocols are also available from commercial vendors, such as Roche Molecular Systems. Furthermore, labels useful in producing probes for use in the disclosed methods are well known in the art and widely available to the skilled artisan. Likewise, methods of incorporating labels into the nucleic acids are also well known to the skilled artisan. As would be apparent to one skilled in the art, primers can be designed, or obtained from vendors, using MLCK polynucleotide sequences such as those of SEQ ID NO: 3 or 4.

Another technique for screening candidate compounds having suitable binding affinity for the MLCK polypeptide is high throughput screening (see International patent application WO84/03564). In this method, large numbers of different small test compounds are synthesized on a solid substrate and then be reacted with the MLCK polypeptide and washed. MLCK polypeptides bound to the candidate compounds may then be detected using methods that are well known in the art (e.g., detection with MLCK specific antibodies). Purified polypeptide can also be coated directly onto plates for use in the aforementioned drug screening techniques.

### Methods of Treatment:

Yet another aspect of the invention pertains to methods of treating heart failure. In this aspect of the invention, candidate compound is administered to a subject in a therapeutically effective amount to activate the MLCK polypeptide.

Another aspect of the invention provides methods of treating heart failure in a subject via gene therapy. In this aspect of the invention, cardiomyocytes are targeted for increased expression of MLCK. Gene therapy can be performed in vivo or ex vivo. Ex vivo gene therapy requires the isolation and purification of patient cardiomyocytes, the introduction of a therapeutic gene and introduction of the genetically altered cardiomyocytes into the patient. In contrast, in vivo gene therapy does not require isolation and purification of a patient's cells.

For in vivo gene therapy, a MLCK gene (for example an autologous MLCK gene isolated from cardiomyocytes the subject or individual or a synthetic MLCK gene obtained from a vendor or other synthesis method) is “packaged” for administration to a subject/individual. Gene delivery vehicles may be non-viral, such as liposomes, or replication-deficient viruses, such as adenovirus as described by Berkner, K. L., in Curr. Top. Microbiol. Immunol., 158, 39-66 (1992) or recombinant adeno-associated virus (rAAV) vectors as described by Muzyczka, N., in Curr. Top. Microbiol. Immunol., 158, 97-129 (1992), Aikawa et al., J. Biol. Chem., 277, 18979-18989 (2002) and U.S. Pat. No. 5,252,479 each of which is hereby incorporated by reference in its entirety. For example, a nucleic acid molecule encoding a MLCK polypeptide can be engineered for expression in a replication-defective retroviral vector or a rAAV vector. The replication-defective retroviral construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo. Alternatively, rAAV vectors comprising the nucleic acid encoding MLCK can be directly introduced into the heart or cardiac tissue in order to effect MLCK expression. Another approach is the administration of “naked DNA” into cardiomyocytes, cardiac fibers or cardiac tissue.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1

Cloning of Cardiac and Skeletal MLCK and Plasmid Construct—RNA isolated from neonatal mouse hearts and skeletal muscle were subjected to reverse transcription using random priming followed by PCR using four sets of specific primers to amplify partial cardiac MLCK cDNA: (fragment 1; F, 5′-CCTTCCAGATGTTAGCACCAAAGTG-3′ R, 5′-TGCCGCACAGCCCTCACCAG-3′), (fragment 2; F, 5′-TGGCAGCACTCCCCCAACC-3′ R, 5′-CCAAACCGACCCCCTCCTAAG-3′), (fragment 3; F, 5′-AAGAGGAGCAGCAACAATGGTG-3′ R, 5′-TTTCAGGCACTGTGTGGCG-3′), (fragment 4; F, 5′-AGTTGGATGTGGTCTTGTTCACG-3′ R, 5′-AAAAGGAAGGGTGCGGGG-3′). Fragments 1-4 were used for cloning of the full length of MLCK cDNA using PCR. Two sets of specific primers were used for amplifying partial skeletal MLCK cDNA: (fragment 1; F, 5′-GACTACAGAAAACGGAGCAGTTGAG-3′ R, 5′-TGCCGCACAGCCCTCACCAG-3′), (fragment 2; F, 5′-AGACACCCAAGGACAAGGAAATG-3′ R, 5′-GGCGGTAGCGAGATGGATTC-3′). HA-tagged partial skeletal MLCK was amplified from fragment 1 with the specific primer set (F, GGGTACCATGTACCCATACGATGTTCCAGATTACGCTACTACAGAAAACGGAGC AGTT, R, 5′-TGCCGCACAGCCCTCACCAG-3′), followed by insertion of fragment 2 into the appropriate restriction sites to construct full length HA-tagged skeletal MLCK.

HA-tagged full length cardiac MLCK was amplified by PCR (F, 5′-GGGTACCATGTACCCATACGATGTTCCAGATTACGCTTCAGGAGTTTCAGAGGAG GA-3′, R, 5′-AAAAGGAAGGGTGCGGGG-3′) and cloned into TOPO-blunt PCR vector. SacI-blunt/KpnI fragment was subcloned into the EcoRI-blunt/KpnI sites of pAdlox shuttle vector (18) to generate pAdlox-cardiac MLCK. KpnI/BamHI-blunt fragment of HA-tagged full length skeletal MLCK was subcloned into the BamHI-blunt/KpnI sites of pAdlox shuttle vector to generate pAdlox-skeletal MLCK.

Adenoviral short-hairpin (sh) RNA was generated according to our standard methods (19). Briefly, the target sites for RNAi of rat cardiac MLCK were designed using the web sites (QIAGEN— http://siRNA.qiagen.com/Index.jsp., and Ambion-http://www.ambion.com/techlib/misc/siRNA_finder.html.m1.). Three specific sites for rat MLCK-RNAi are: (1) CTTAATGTGCTGACTGAGA, (2) CAGATGCAGAGACCATGAA, (3) ATATATGGCTCAGCGTAAA. Rat NRx2-5-RNAi target sequence is GGCGGTGGAGCTGGACAAA. Scrambled sequences with no homology to known genes were used for control RNAi.

GST-MLCK (28 to 463 aa) vector was generated by insertion of NcoI/XbaI fragment of MLCK cDNA into NcoI/XbaI site of pGEX-CD vector. GST-MLC2V vector was generated by insertion of the BamHI/EcoRI fragment of pcDNA3-rat MLC2V into BamHI/EcoRI site of pGEX-CD vector. GST-MLC2A vector was generated by insertion of the HindIII/SalI digested fragment of the PCR products using two specific primers for mouse MLC2A (F′ 5′-GAAGCTTCATGGCCAGTAGGAAGGCTGGG-3′; R, 5′-CGTCGACCTACTCCTCTTTCTCATCCCCGTG-3′) amplified from the cDNA (IMAGE Consortium CloneID 30300846, OPEN BIOSYSTEMS) (20).

Cardiomyocyte Preparation, Adenovirus Infection and Measurement of Cell Shortening and Simultaneous Intracellular Free calcium—Neonatal rat ventricular cardiomyocytes were isolated from hearts of 2-day-old Sprague Dawley rats by trypsin digestion followed by Percoll gradient purification according to a protocol described previously with some modifications (21). Infection of adenovirus-shRNA (for MLCK and NRx2-5, 100 moi) was performed in suspension immediately after purification for 2 hrs followed by plating on plates or laminin-coated glass coverslips for 48 to 96 hrs in medium (DMEM F-12, 5% new born calf serum, 0.5% of Insulin-Transferrin-Selenium) (Gibco-Invitrogen). Adenovirus Adlox-MLCK (10 and 50 moi) and control Adlox-beta-galactosidase (50 moi) were infected for 2 hrs 48 hrs after plating followed by an additional 24 to 48 hr incubation.

Measurement of cell shortening and simultaneous intracellular free calcium were performed according to methods described previously with some modifications (22). In brief, myocytes were loaded with the acetoxymethyl ester of fura-2 (0.1 μmol/L, Molecular Probes/Invitrogen) in Tyrode's solution (mmol/L: NaCl 137, KCl 3.7, NaH₂PO₄ 1.2, CaCl₂ 1.2, HEPES 20, MgSO₄ 1.2, glucose 15; and 0.0005% Pluronic F-127) for 10 minutes at room temperature. Myocytes were rinsed with Tyrode's solution and maintained for 20 minutes at room temperature to allow for de-esterification of the dye, followed by superfusion with modified Tyrode's solution (mmol/L: NaCl 137, KCl 3.7, NaH₂PO₄ 1.2, CaCl₂ 1.2, HEPES 4, MgCl₂ 0.5, glucose 15, probenesid 1.0, pH 7.4) on a temperature-controlled chamber (32° C.) mounted on an Olympus inverted microscope. When studied at two Ca²⁺ concentrations, myocytes were sequentially superfused with 1.2 mM Ca²⁺ followed by 2.5 mM for 30 minutes before measurement. A dual excitation spectrofluorometer was used to record fluorescence emissions (505 nm) elicited from exciting wavelengths at 340 and 380 nm. Myocytes were imaged with a CCD video camera attached to the microscope and motion was quantified by a video motion detector system (IonOptix). To provide high-contrast spots for tracking cell motion of neonatal cardiomyocytes, glass beads 2±0.5 μm (Duke Scientific Corp) were added as described previously (23). Results between groups were compared using ANOVA and Fisher PLSD post-hoc test. Statistical tests were performed using StatView version 5.01; p<0.05 was considered significant.

Animal Models—Floxed-NRx2-5 mice (24) were bred with transgenic mice carrying the Cre-ER™ gene under CMV promoter (25). Matings generated mice homozygous or heterozygous for the floxed-NRx2-5 gene with or without being heterozygous for Cre-ER™ transgene. Female mice homozygous or heterozygous for floxed-NRx2-5/Cre-ER™ (−) were bred with male mice homozygous or heterozygous for floxed-NRx2-5/Cre-ER™ (+) to generate floxed/floxed/Cre-ER™ (+), floxed/floxed/Cre-ER™ (−) or wild/wild/Cre-ER™ (+). To delete the floxed-NRx2-5 gene, tamoxifen (0.5-1 mg/g body weight, ip) was injected into pregnant mice within 24 hr before delivery. All animal care protocols fully conformed to the Association for the Assessment and Accreditation of Laboratory Animal Care, with approvals from the University of Florida Institutional Animal Care and Use Committees. Upper-septum of the hearts isolated from post-myocardial infarction mice previously described (26) was utilized for Western blotting to examine cardiac MLCK and GAPDA expression.

GST Fusion Protein and MLCK Antibody Production—GST fusion proteins were expressed in E. coli BL21(DE3) (Stratagene) with 1 mM of IPTG for 4 hr at 37 C. Bacteria were lysed by sonication in lysis buffer [20 mM HEPES, pH7.5, 100 mM NaCl, 10 mM MgC1, 1% Triton x-100, proteinase inhibitor cocktail (complete, Roche)]. Lysates were incubated with Glutathione Sepharose (Amersham) and washed with lysis buffer 5 times. For in vitro kinase assays, GST proteins bound to Glutathione Sepharose were further washed with kinase buffer (25 mM HEPES, pH7.6, 200 mM NaCl, 10 mM MgCl₂) three times. GST-fusion proteins were eluted with 10 mM of reduced Glutathione (Sigma) in the kinase buffer.

GST-MLCK proteins bound to Glutathione Sepharose were washed with PBS, followed by cleavage of GST protein using thrombin (20 U/ml) for 3 hrs. After addition of Tris-HCl (final concentration 50 mM) and NaCl (final concentration 0.5 M) thrombin was removed by Benzamidine Sepharose (Amersham). MLCK proteins were further purified by SDS-PAGE followed by electro-elution. The purified proteins (150 μg) were emulsified either with complete Freund's adjuvant (day 1) or incomplete Freund's adjuvant (days 14, 28, and 42) to immunize New Zealand White rabbits for polyclonal antibody production. Affinity purification of antibodies was performed using GST-MLCK fusion protein (28-463 aa) covalently bound to agarose beads using AminoLink Plus Immobilization Kit (PIERCE). Northern and Western Blotting and Immunostaining—Northern blot analyses were performed using the following probes: cardiac MLCK RT-PCR product (1266 bp, F: 5′-TGGCAGCACTCCCCCAACC-3′, R: 5′-CCAAACCGACCCCCTCCTAAG-3′); mouse skeletal MLCK RT-PCR product (552 bp, F: 5′-GACTACAGAAAACGGAGCAGTTGAG-3′, R: 5′-GGGTCAGGGGTCACAGACACC-3′); NRx2-5 probe, PflMI-EcoRI fragment of mouse NRx2-5 cDNA.

Western blot analyses and immunostaining were performed with the following primary antibodies: anti-HA (clone 3F10, Roche), anti-MLC2 (F109.3E1, BioCytex, Marseille, France), anti-MLCK pAb described above, anti-phospho-serine (29675, AnaSpec), anti-phospho-tyrosine (4G10, Upstate) and anti-troponin T (T6277, SIGMA). Rhallodin-TRITC (77418, Sigma) was utilized for detecting polymeric F-actin. Fluorescent microscopic images were obtained usings ZEISS Axiovert200M with or without Apotome.

Phosphorylation Assays—Phosphorylation assays were performed according to protocols described previously with some modifications (27). Briefly, in vitro phosphorylation reactions were performed using recombinant HA-tagged full-length MLCK proteins expressed by adenovirus in 293 cells. Immunoprecipitated MLCK proteins using anti-HA antibody (3F10, Roche) bound to protein G sepharose (Sigma) were washed three times with kinase buffer, mixed with GST, GST-MLC2v or GST-MLC2a fusion protein (approximately 2 μg) in the presence of 1 μl of [γ-³²P]ATP (0.25 mCi/ml) in 20 μl of kinase buffer at 37° C. for 30 min. Immunoprecipitants without anti-HA antibodies were used as negative controls. 10 μl of each samples were boiled in SDS-PAGE sample buffer and subjected to SDS-PAGE. Kinase activity was measured as the rate of ³²P incorporation into GST-MLC2v fusion protein. HA tagged-cardiac MLCK and HA-tagged skeletal MLCK expressed in 293 cells were purified by HA-affinity columns and eluted with HA-peptides (Roche). GST-MLC2v and HA-tagged cardiac and skeletal MLCK proteins were dialyzed against protein lysis buffer (25 mM HEPES, PH7.6, 100 mM NaCl, 5 mM MgCl₂, 1 mM DTT, 1% triton, 10% glycerol, protease inhibitor cocktail). A 1:10 dilution of these proteins were utilized in the kinase reaction (approximately cardiac MLCK 0.15 μg/ml, 1.7 nM, skeletal MLCK 0.15 μg/ml, 2 nM, GST-MLC2v 0.14 mg/ml, 3 mM with 2 fold serial dilution) in 25 μl of kinase buffer (final concentration 25 mM HEPES pH7.6, 10 mM MgCl₂, 5 mM DTT, 20 mM NaCl, 0.2% triton, 2% glycerol, 0.5 mg/ml BSA, 0.5 mM [γ-³²P]ATP at 267 cpm/pmol) at 30° C. for 15 min. Either 1 mM Ca²⁺ and 1 μM calmodulin (Sigma P2277) or 5 mM EGTA were added in the kinase reaction to examine Ca²⁺/calmodulin dependence. After termination of the kinase reaction by the addition of SDS-sample buffer, the samples were separated by 15% SDS-PAGE gel, stained with Coomassie blue and autoradiographed. Phosphorylated MLC2v proteins were excised from the gel and their radioactivity was measured using a liquid scintillation counter. Km and Vmax of cardiac MLCK were determined using Lineweaver-Burk analysis (Prism 4, GraphPad Software, Inc. San Diego, Calif.)

For detection of ³²P-labeled MLC2v, neonatal ventricular cardiomyocytes (2×10⁵ cells/3 cm plate) infected with adenovirus were labeled with [³²P]-orthophosphate at 0.1 mCi/ml in phosphate-free DMEM containing 10% dialyzed FBS (GIBCO/Invitrogen) for 1 hr. Cells were lysed with 0.6 ml of RIPA buffer (50 mM Tris, pH 7.5, 150 mM NaCl, 0.1% SDS, 1% NP40, 2 mM EDTA, 1% deoxycholate, protease inhibitor cocktail, 10 mM NaF, 2 mM Na₃VO₄). 0.25 ml of the lysates was pre-cleared with protein G, and then immunoprecipitated with anti-MLC2 antibody coupled to protein G. Immunoprecipitants without anti-MLC2 antibodies were used as negative controls. After washing with RIPA buffer (5 times), the samples were transferred to PVDF membrane (Millipore) after SDS-PAGE and detected with autography. The membrane was then immunoblotted with anti-MLC2 antibody for detection of the total MLC2v proteins. The relative amount of phosphorylated and total MLC2v was determined by densitometric analyses.

### Results

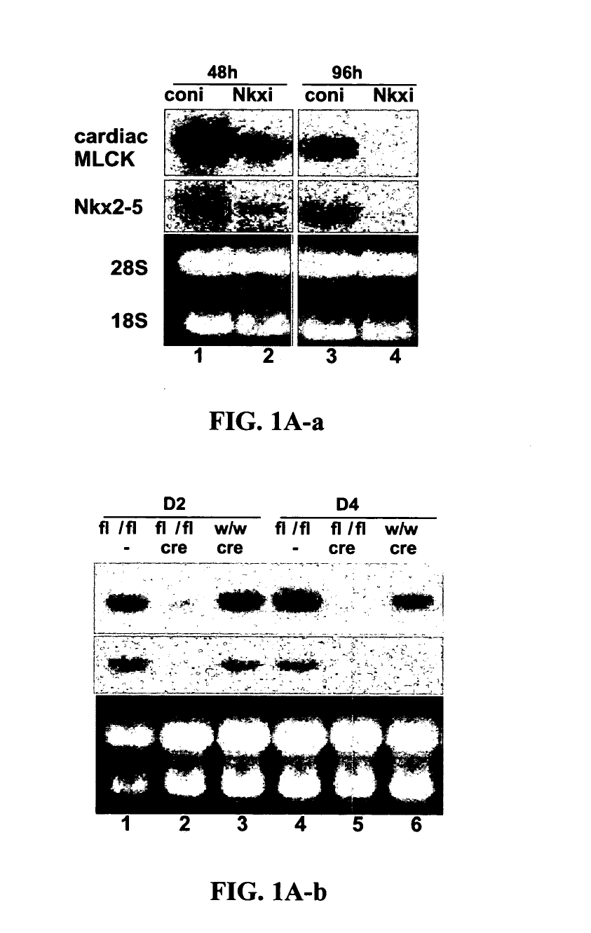

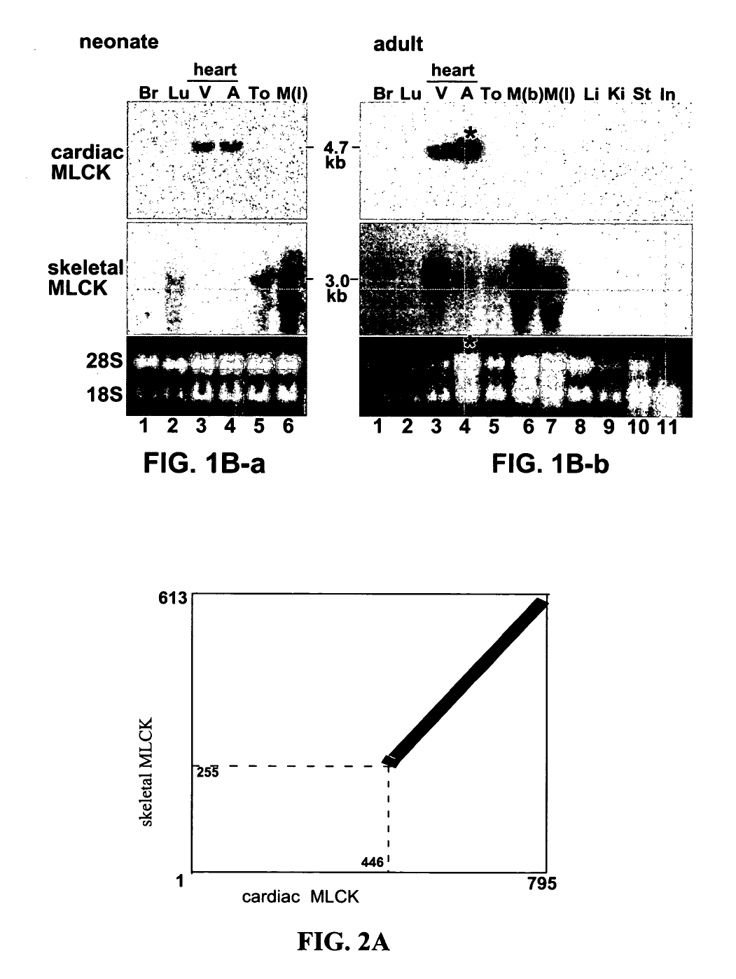

Identification of a Cardiac-Specific MLCK as a Downstream Target of Cardiac Homeobox Protein, NRx2-5—In the process of identifying downstream targets of the cardiac homeobox transcription factor, NRx2-5 in postnatal hearts, a gene product homologous to skeletal and smooth muscle MLCK (NCBI UniGene No. Rn.43838-rat and Mm32804-mouse) was identified. In NRx2-5 knocked-down neonatal rat cardiomyocytes (FIG. 1A-a), as well as inducible NRx2-5 knockout hearts (FIG. 1A-b), reduced expression of NRx2-5 dramatically decreased mRNA expression of the MLCK homologue, indicating that NRx2-5 regulates its expression either in a direct or indirect manner. Because NRx2-5 expression is nearly restricted to cardiac myocytes at the postnatal stage (28-30), tests as to whether the expression of MLCK homologue is also cardiac-specific were performed. Multi-tissue Northern blotting readily detected mRNA of the MLCK homologue specifically in the heart in both ventricle and atrium at neonatal (FIG. 1B-a, top panel) and adult stages (FIG. 1B-b, top panel). Hybridization of the same membrane with the skeletal MLCK-specific probe revealed that the expression of skeletal MLCK is below the level of detection in the neonatal heart (FIG. 1B-a, middle panel), and only weakly detected in adult ventricle (FIG. 1B-b, middle panel). Therefore, the MLCK homologue is hereafter called cardiac MLCK.

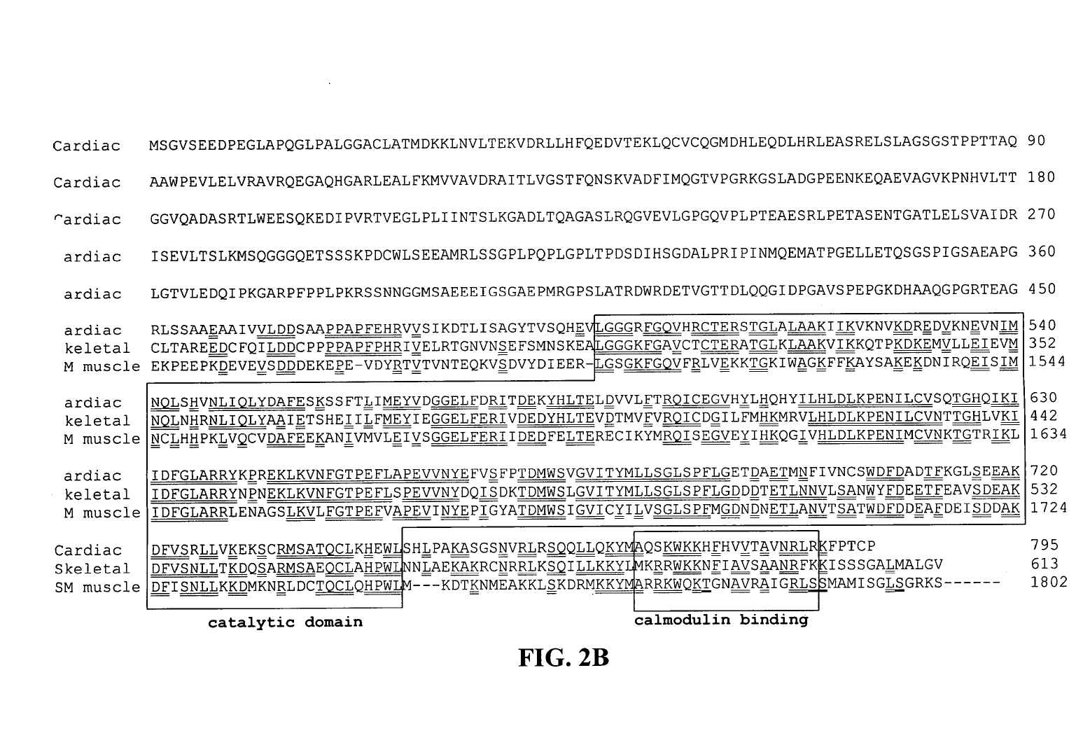

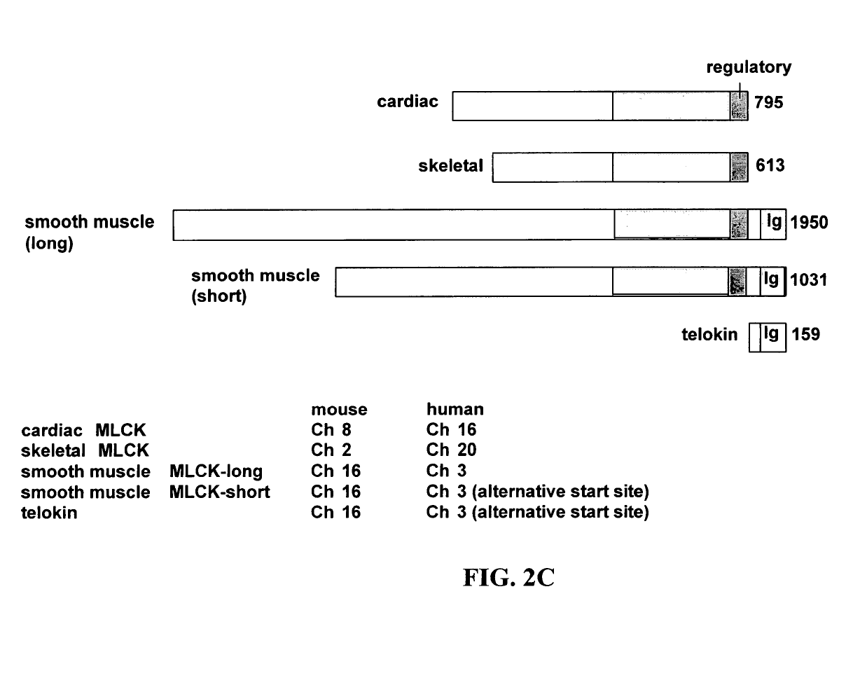

The cardiac MLCK gene encodes 795 amino acids with a predicted MW of 86 kDa excluding post-translational modifications. The protein consists of a conserved kinase domain at the carboxyl-terminus with a 58% identity with skeletal MLCK and 44% identity with smooth muscle MLCK. However the amino-terminal domain is quite unique with no significant homology to other known proteins including skeletal MLCK (FIG. 2A). The amino acid sequence alignment of cardiac MLCK to skeletal MLCK and long-form smooth muscle MLCK is shown in FIG. 2B. Characterization of this cardiac MLCK results in having three MLCKs: cardiac-, skeletal-(encoded by mylk1) and smooth-muscle (encoded by mylk2), with conserved catalytic and kinase regulatory domains (Ca²⁺/calmodulin-binding domain) at the carboxyl-terminus, with the exception of the shortest form of smooth muscle MLCK, telokin (FIG. 2C).

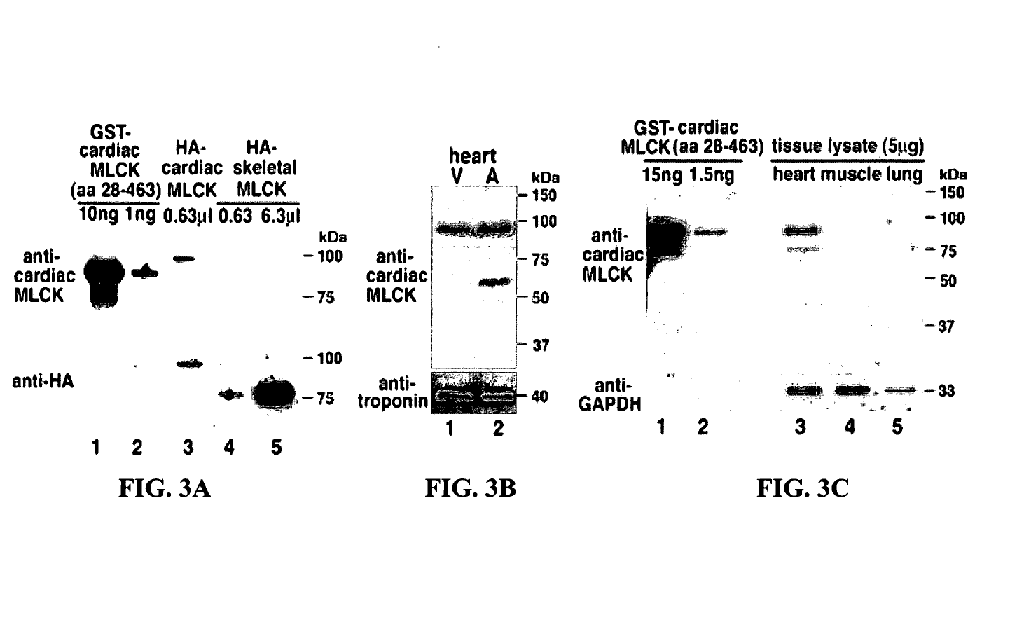

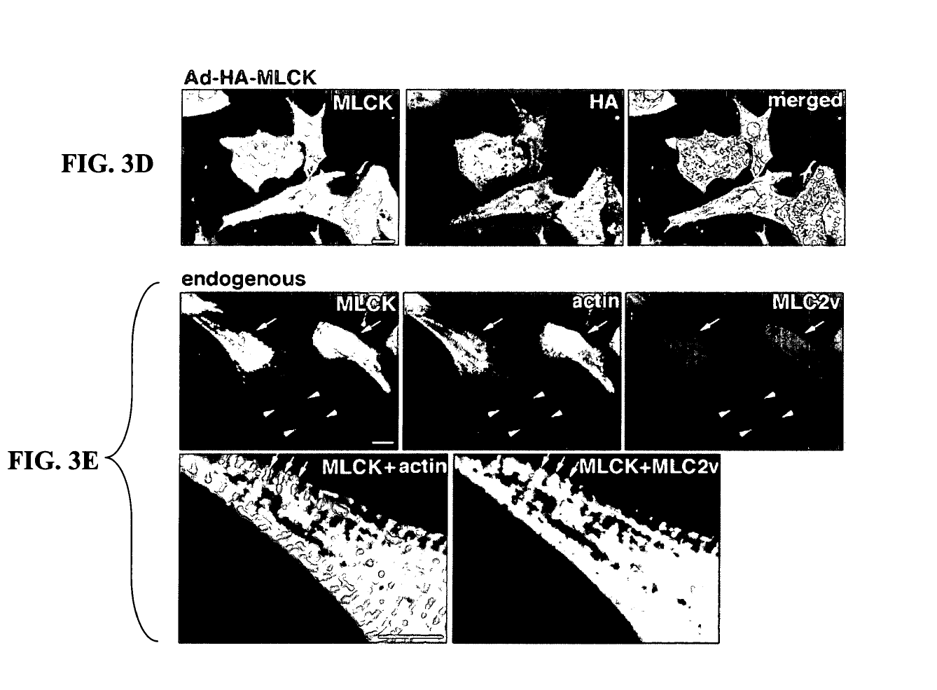

Cardiac MLCK Protein Expression—Affinity-purified antibody against the unique amino-terminus domain of cardiac MLCK(28-463 aa) (FIG. 3A, lane 1, 2) was made. The specificity of the antibody was confirmed by its reactivity to HA-tagged full-length cardiac MLCK (FIG. 3A, lane 3), but not to HA-tagged full-length skeletal MLCK even at 10-fold abundance (FIG. 3A lanes 4, 5). Of note, cardiac MLCK has a higher homology to skeletal. MLCK than to smooth muscle MLCK (see FIG. 2). In neonatal heart lysates, cardiac MLCK protein of approximately 90 kDa was readily detected in both ventricle and atrium at similar expression levels (FIG. 3B). Cardiac MLCK protein expression in the heart lysates is relatively abundant (equivalent to 2.3 ng of GST-cardiac MLCK in 5 μg of heart lysates, estimated concentration in our experimental condition 0.45 μg/mg) (FIG. 3C), which is lower but comparable to skeletal MLCK expression in skeletal muscle previously reported (6). The antibody against amino-terminus domain of cardiac MLCK does not appear to cross-react with other proteins in the tissue lysates of skeletal muscle and lung in which skeletal or smooth muscle MLCK is abundantly expressed (6). These results indicate that cardiac MLCK protein is selectively and relatively abundantly expressed in the heart and is consistent with the mRNA expression shown in FIG. 1B.

To examine the intracellular localization of MLCK, HA-tagged MLCK was expressed by adenovirus delivery and detected ectopic MLCK using anti-HA (red, FIG. 3D) and anti-MLCK antibodies (green, FIG. 3D) in ventricular neonatal cardiomyocytes. Punctuated MLCK staining appears diffusely localized in the cytoplasm when MLCK was overexpressed (FIG. 3D). The merged image clearly demonstrated colocalization of HA-tagged MLCK using anti-HA and anti-MLCK antibodies (FIG. 3D). Endogenous cardiac MLCK staining is largely diffusely localized in the cytoplasm of cardiomyocytes as well (FIG. 3E, green, arrows. Arrowheads: non-cardiomyocyte); however, in some areas, striated staining of cardiac MLCK was observed. To examine whether cardiac MLCK is co-localized with MLC, the only known substrate for skeletal and smooth muscle MLCK, cardiomyocytes were co-immunostained to detect actin (FIG. 3E, red, localizing at I bands) and MLC2v (FIG. 3E, blue, localizing at A bands). Interestingly, the enlarged images demonstrate that striated MLCK staining was co-localized with actin (FIG. 3E, green and red) but not with MLC2v (FIG. 3E, green and blue). Skeletal and smooth muscle MLCK catalytic activity is known to be enhanced by Ca²⁺/calmodulin binding to the regulatory domain located within the carboxyl terminus (FIG. 2B), which results in conformational changes in the catalytic domain. Thus, the ability of added calcium ionophore A23187 to induce translocation of MLCK within close proximity to MLC2v was examined; however, there was no observation of significant changes in cardiac MLCK localization after addition of A23187 between 15 min to 2 hr (data not shown).

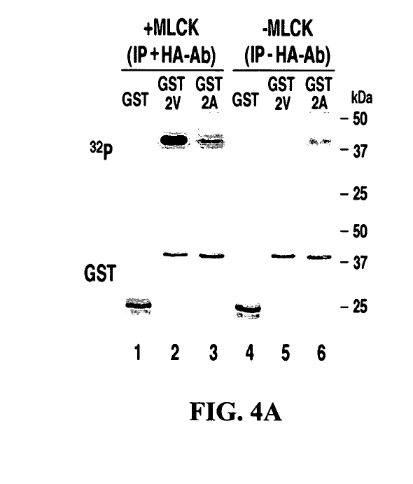

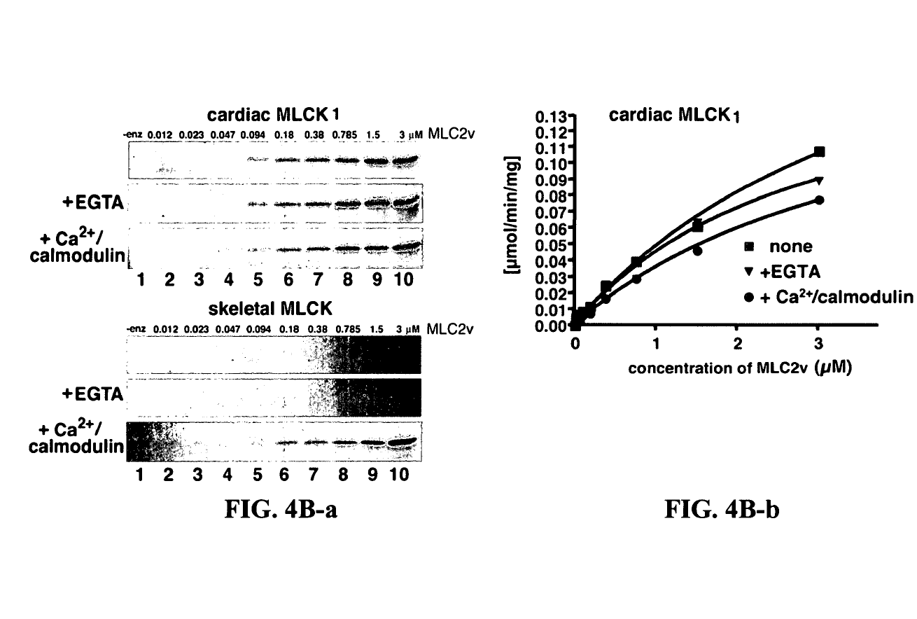

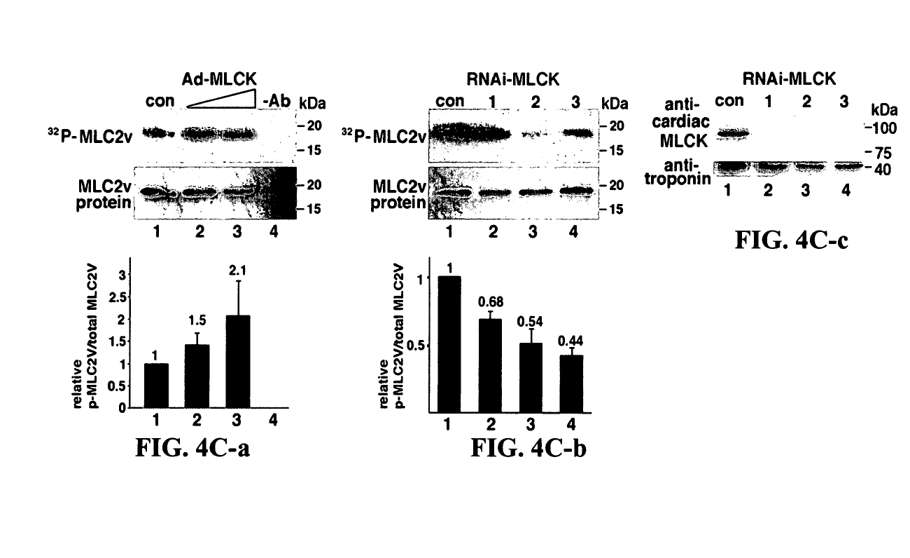

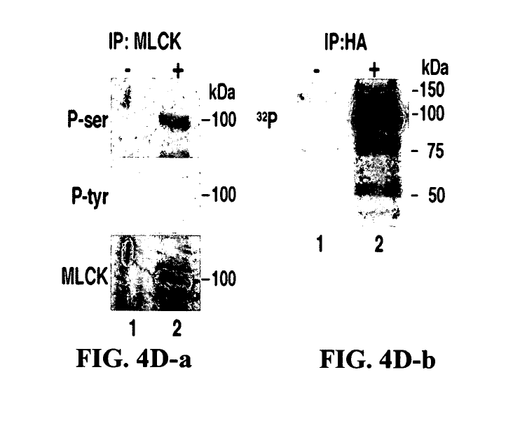

Cardiac MLCK Phosphorylates MLC2 in Vitro and in Vivo as Well as Potentially MLCK Itself—The ability of cardiac MLCK to phosphorylate its putative substrate MLC2 was also examined. In vitro kinase assays using HA-tagged cardiac MLCK expressed in 293 cells promotes incorporation of ³²P to GST-MLC2v and GST-MLC2a fusion proteins (FIG. 4A). This result indicates that ectopically expressed cardiac MLCK in 293 cells is sufficient for phosphorylation of MLC2v and MLC2a in vitro. Catalytic activity of skeletal- and smooth muscle MLCKs is Ca²⁺/calmodulin dependent. However, cardiac MLCK phosphorylated MLC2v in the absence of Ca²⁺/calmodulin (FIG. 4B-a, top panels-cardiac MLCK). Furthermore, addition of EGTA (FIG. 4B-a, top panels, +EGTA), or Ca²⁺/calmodulin (FIG. 4B-a, top panels, +Ca²⁺/calmodulin) had little effect on the catalytic activity of cardiac MLCK, while it greatly affected skeletal MLCK's catalytic activity (FIG. 4B-a, bottom panel-skeletal MLCK). The estimated kinetic constant determined by Lineweaver-Burk plot was Km value of 4.3±1.5 μM and Vmax 0.26±0.06 μmol/min/mg (without EGTA and Ca²⁺/calmodulin). The low Km value of cardiac MLCK indicating high affinity to the substrate is equivalent to skeletal MLCK to skeletal MLC (3.5 μM) and smooth muscle MLCK to smooth muscle MLC (6-11 μM) (9,10).

Next, an assay was performed to determine whether cardiac MLCK affects the level of MLC2v phosphorylation in cultured ventricular cardiomyocytes. Overexpression of cardiac MLCK increases MLC2v phosphorylation nearly 2.1 fold in a dose-dependent manner (FIG. 4C-a), which is likely near the level of saturation based on the previous report that 30 to 40% of total MLC2v is phosphorylated in steady state conditions (31). Conversely, decreased MLCK expression by infection of three MLCK-shRNA adenoviruses targeting different MLCK sequences decreases MLC2v phosphorylation levels by 30-55% (FIG. 4C-b). Notably, incorporation of [³²P]-orthophosphate is reduced by, at most, 55% by MLCK-RNAi; however cardiac MLCK expression was nearly below the detection level (FIG. 4C-c).

Catalytic activities of smooth muscle and skeletal MLCKs are known be regulated by phosphorylation predominantly by upstream kinases (32). Cardiac MLCK also appears to be phosphorylated at serine residue(s) but not at tyrosine residues (FIG. 4D-a). Incorporation of ³²P to cardiac MLCK itself was detected in an in vitro kinase assay in which immunoprecipitated exogenous cardiac MLCK proteins expressed in 293 cells were utilized (FIG. 4D-b). This result suggests that either MLCK autophosphorylates MLCK, or the presence of MLCK upstream kinases physically interact with cardiac MLCK in 293 cells.

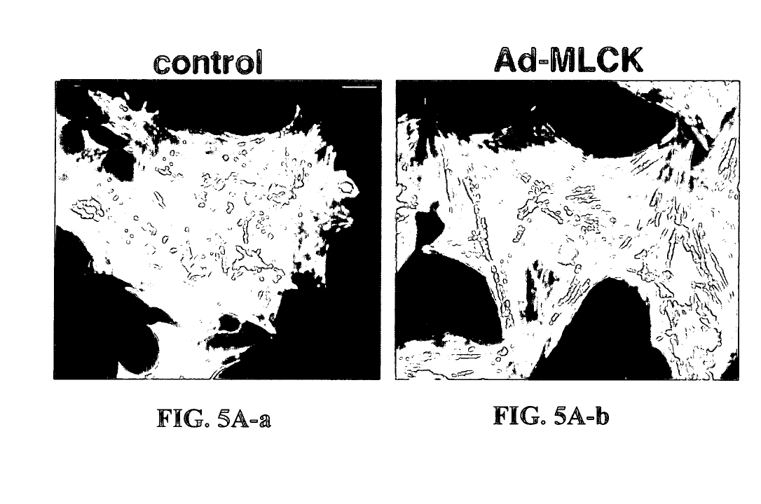

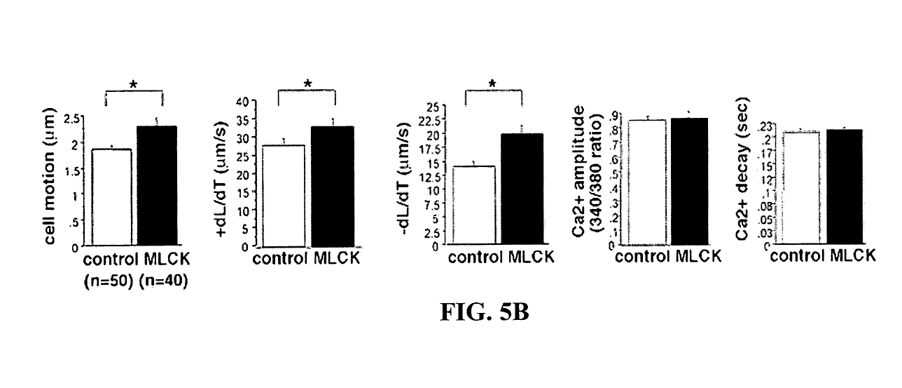

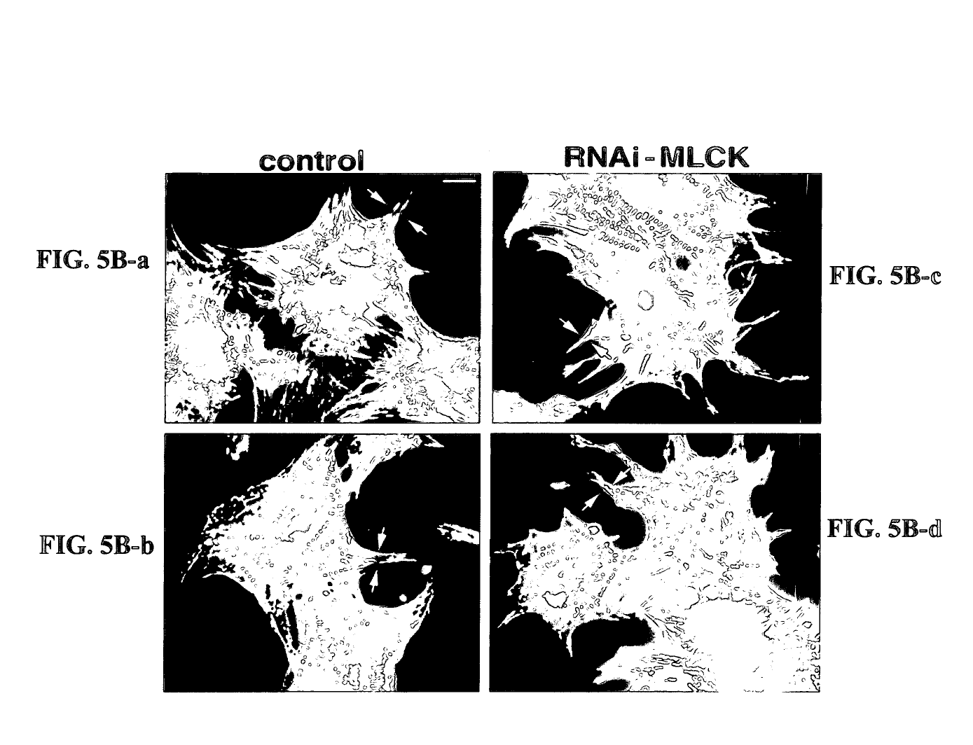

Cardiac MLCK Promotes Sarcomere Organization and Increases Cardiomyocyte Contractility—It was observed that cardiomyocytes in which cardiac MLCK was overexpressed displayed organized sarcomere structures characterized by straight, thick, striated actin bundles detected by phalloidin (FIG. 5A, a vs. b). This indicates that increased expression of cardiac MLCK is sufficient to induce sarcomere organization. However, in cardiomyocytes in which cardiac MLCK expression levels were reduced using RNAi, striated sarcomere structure was not apparently affected, with only slight changes in peripheral structure up to 96 hrs after adenoviral infection (FIG. 5B, a, b vs. c, d).

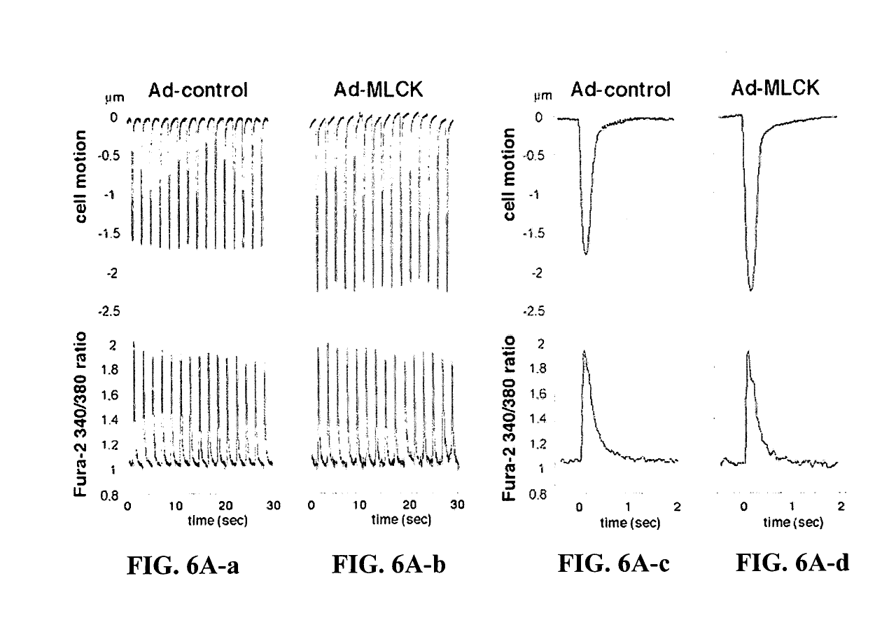

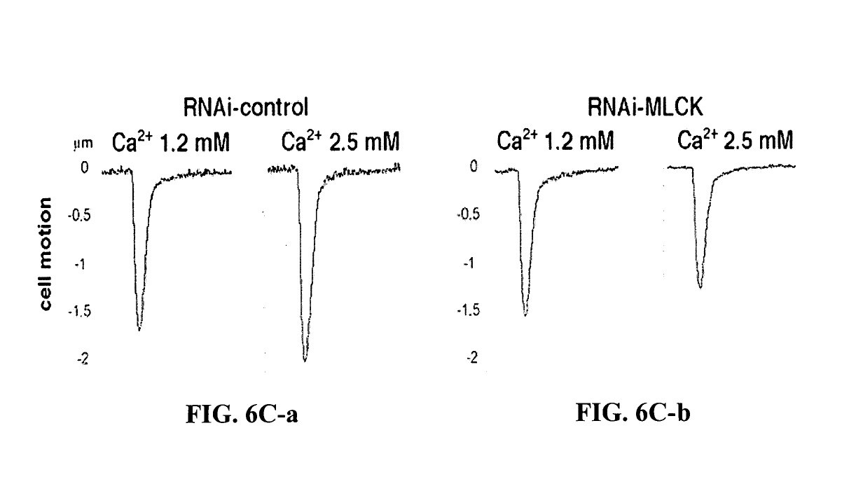

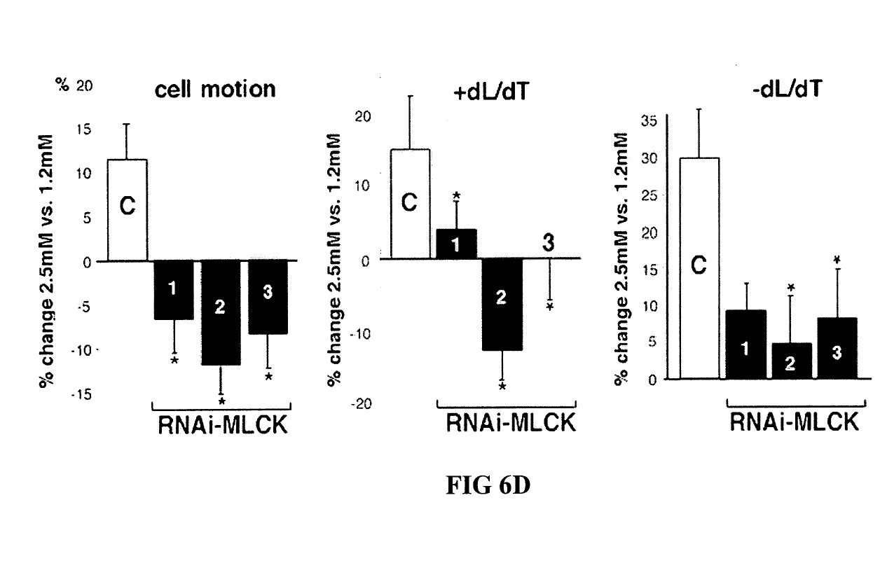

Experiments that examined whether cardiac contraction is affected by increased or decreased MLCK expression by measuring cardiomyocyte cell shortening and simultaneous intracellular Ca²⁺ transients were performed. These experiments showed that adenoviral-mediated overexpression of MLCK resulted in significantly increased cardiomyocyte contraction amplitude, and kinetics of contraction and relaxation without a significant change in intracellular Ca²⁺ transients (FIG. 6A, a vs. b and c vs. d; FIG. 6B).

On the other hand, MLCK knockdown did not significantly affect cardiomyocyte contraction at the standard Ca²⁺ superfusate concentration (1.2 mM) (FIG. 6C, Ca²⁺ 1.2 mM, a vs. b, Table 1). In control cardiomyocytes, increasing Ca²⁺ concentration to 2.5 mM enhanced cardiac contraction by nearly 11% compared to the basal 1.2 mM Ca⁺ concentration (FIG. 6C-a). However, this positive effect was not observed in MLCK-knockdown cardiomyocytes, and rather cell contraction was reduced by increased Ca²⁺ concentration (FIG. 6C-b). Compared to control-RNAi treated cardiomyocytes, MLCK knocked-down cardiomyocytes exhibited significantly reduced responses to increasing Ca²⁺ concentration in contraction amplitude, and kinetics of contraction and relaxation with the exception of one parameter [−dL/dT in MLCKi(1)] (FIG. 6D, Table 2), without significant change in intracellular Ca²⁺ transients (data not shown).

In summary, overexpression of cardiac MLCK potentiates cardiomyocyte contraction, but reduced expression of cardiac MLCK does not affect cardiac contraction at the basal Ca²⁺ concentration. Reduction of cardiac MLCK affects cardiomyocyte contraction when extracellular Ca²⁺ concentration was increased, when greater MLCK activity might be required to achieve higher level of cardiac contraction.

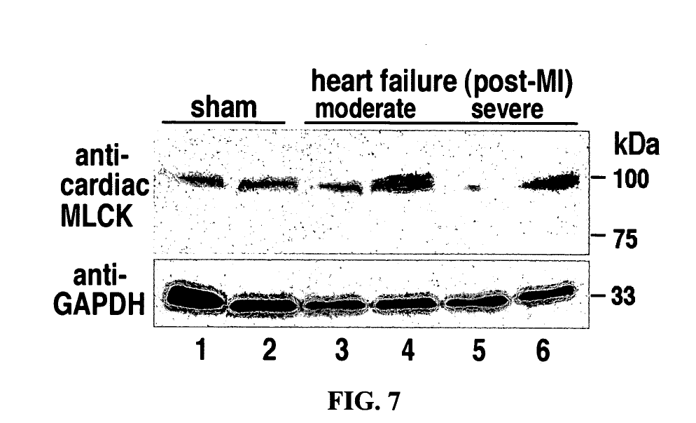

Cardiac MLCK Expression in Post-myocardial Infarction Heart Failure Mouse Model—To investigate whether cardiac MLCK protein expression is altered in heart failure, cardiac tissue from mice with heart failure due to coronary artery ligation was examined. Cardiac MLCK expression in the non-infarcted upper ventricular septum was significantly downregulated in mice with severe heart failure compared with sham-operated mice (FIG. 7, lanes 1, 2 vs. lanes 5, 6). These results support the findings in cultured cardiomyocytes (FIG. 6D).

Cardiac MLCK Expression and MLC2v Phosphorylation in Mice with NRx2-5 Knockout, Aging and Post-Myocardial Infarction Heart Failure.

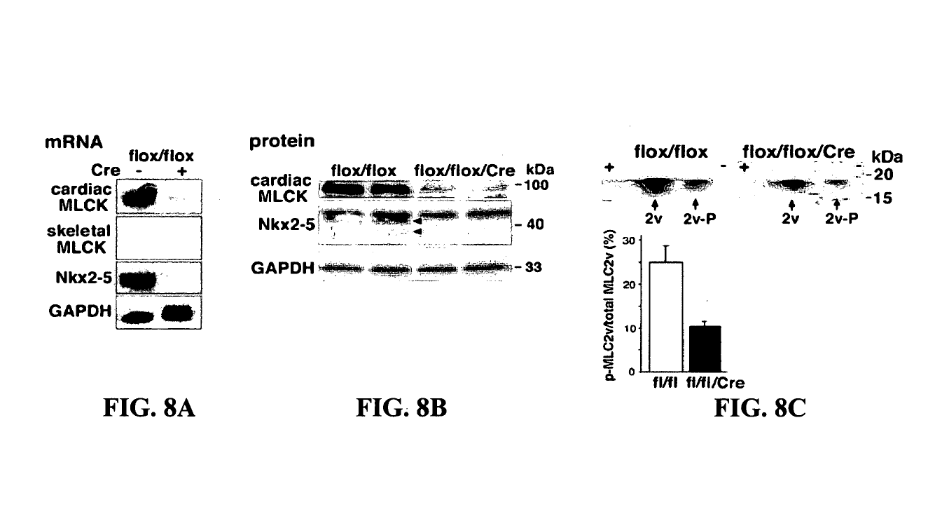

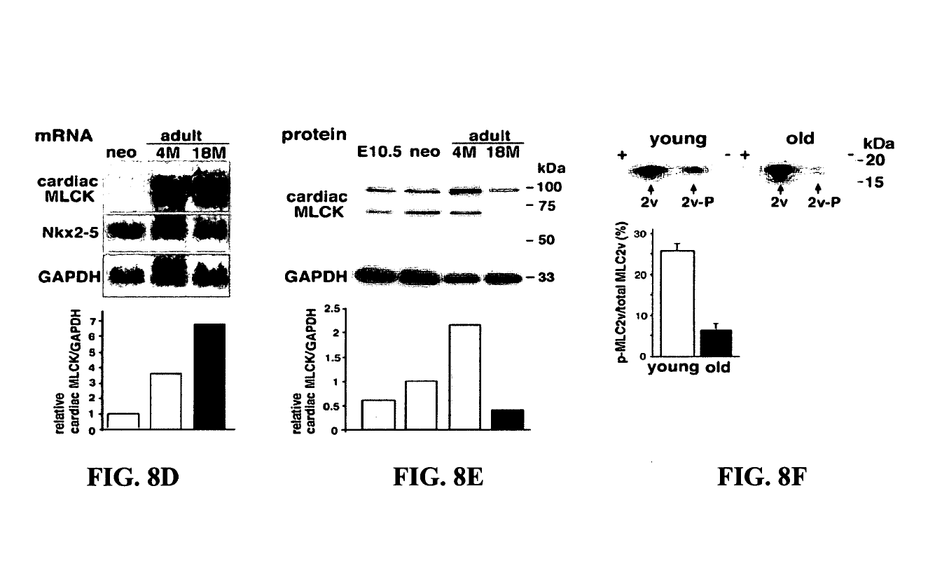

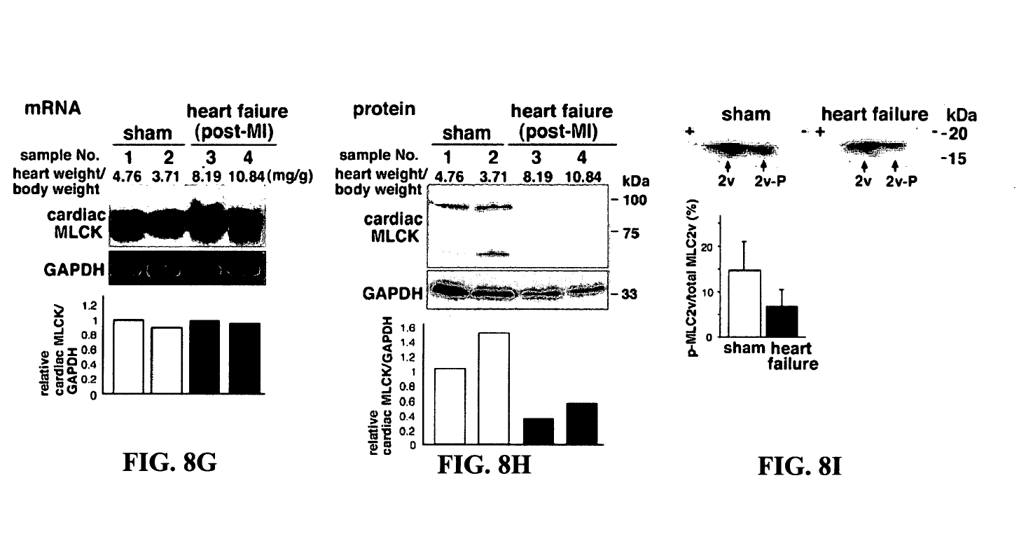

To examine catalytic activities of cardiac MLCK in vivo, we first examined cardiac MLCK expression and MLC2v phosphorylation levels in NRx2-5 knockout hearts at postnatal day 12 when expression of cardiac MLCK mRNA (FIG. 8A) and protein (FIG. 8B) were markedly reduced. Skeletal MLCK mRNA expression in NRx2-5 knockout hearts was below the level of detection by Northern blotting (FIG. 8A, skeletal MLCK); however using quantitative real-time PCR, skeletal (2.55±0.04 fold, n=2) and smooth muscle MLCK (1.48±0.02 fold, n=2) expression were increased. Despite this apparent compensatory increase, the level of MLC2v phosphorylation was decreased in hearts from NRx2-5 knockout mice by nearly 60% compared to age-matched control mice (FIG. 8C).

Cardiac MLCK mRNA increased during development from neonatal to adult stages and persisted in the aged hearts (FIG. 8D). Of note, greater separation of RNA by agarose gel electrophoresis revealed two hybridized bands near 4.7 kb with similar intensities. Cardiac MLCK protein increased in hearts from embryonic day 10.5, neonates and adult, but was decreased in aged hearts (FIG. 8E). Consistent with decreased cardiac MLCK protein in aged hearts (18 and 21 months old), MLC2v phosphorylation was decreased in aged hearts compared to postnatal day 12 hearts (FIG. 8F).

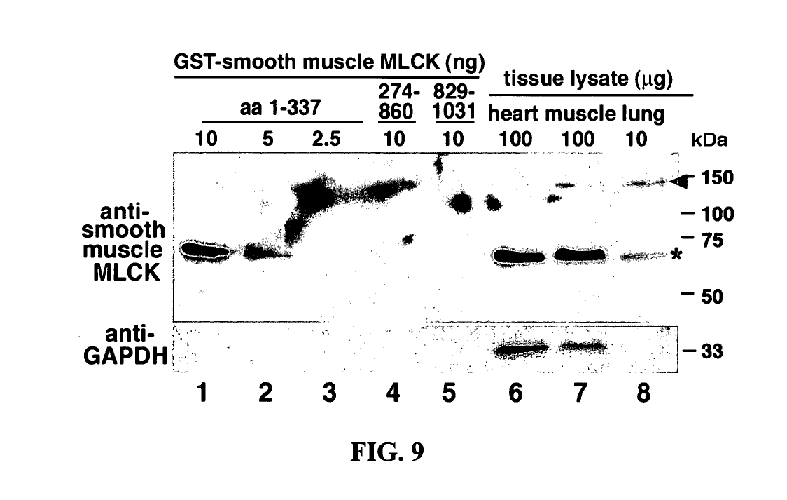

We next examined cardiac MLCK expression in a post-myocardial infarction mouse model of heart failure three weeks after coronary artery ligation. At the mRNA level, cardiac MLCK expression in non-infarcted upper ventricular septal tissue was similar to tissue from sham-operated age-matched mice (FIG. 8G, lanes 1, 2 vs. lanes 3, 4). In contrast, cardiac MLCK protein was decreased in heart failure tissue compared to control tissue (FIG. 7H, lanes 1, 2 vs. lanes 3, 4). Furthermore, levels of MLC2v phosphorylation were decreased in heart failure compared to controls (FIG. 8I). The lack of concordance between mRNA vs. protein levels in neonatal and aged hearts and in failed hearts suggests altered posttranscriptional regulations of cardiac MLCK in aging and heart failure. Additionally, we confirmed the expression of 130-kDa short-form smooth muscle MLCK in the heart⁶ (estimated concentration 0.025-0.05 μg/mg), skeletal muscle, and more abundantly in the lung (0.5-1 μg/mg) (FIG. 9). Thus, cardiac MLCK protein expression is approximately 10-20 fold more abundant than smooth muscle MLCK in the neonatal heart.

Skeletal and smooth muscle MLCKs are known to preferentially phosphorylate their tissue-specific MLC2 substrates. For instance, smooth muscle MLCK has a high affinity to smooth muscle MLC, but not to skeletal- and cardiac MLC2v (8-10). Although MLC2 phosphorylation is critical for the regulation of cardiomyocyte contraction (5), a cardiac-specific MLCK has not yet been demonstrated. In this study, a homologue of skeletal and smooth muscle MLCK that is preferentially expressed in the heart, herein named cardiac MLCK, was isolated and localized to cardiac tissue. Despite the availability of sequence data of this cardiac MLCK in the public domain, this gene and its protein have not been characterized. Expression of cardiac MLCK mRNA is markedly downregulated shortly after reduction of NRx2-5 expression by NRx2-5 knockdown and inducible NRx2-5 knockout. NRx2-5 expression is nearly restricted to the heart in the postnatal stage (28-30), and its downstream target gene cardiac MLCK expression is detected only in the heart using multi-tissue Northern blotting. A recent study showed that genes regulated in both heart failure patients and in murine models of heart failure, contain significantly more putative NRx2-5 binding sites in their 5 kb promoter region (33). Thus, our data may shed light on one potential NRx2-5-dependent regulatory pathway through regulation of cardiac MLCK expression in heart failure. Either direct- or indirect-effects of NRx2-5 on cardiac MLCK transcription remain to be elucidated.

Increased expression of cardiac MLCK induces organized sarcomere structure in neonatal cardiomyocytes characterized by straight, thick, striated actin bundles as seen by overexpression of skeletal MLCK (12). Ser19 phosphorylation of MLC2 leading to potentiation of the force and speed of contraction has been well studied in smooth muscle and skeletal muscle (3,32). Our experiments also demonstrated that overexpression of cardiac MLCK enhances cardiomyocyte contraction, likely due to a combination of increased MLC2 phosphorylation and organized sarcomere structure stably formed within 36-48 hr after MLCK adenovirus infection. Of note, cultured neonatal cardiomyocytes are used frequently because of their unique rapid modification of sarcomere organization and overall cellular morphology (34).

On the other hand, decreased expression of cardiac MLCK did not markedly disturb sarcomere organization within 96 hrs. Consistent with the subtle morphological changes, myocytes in which MLCK was knocked-down did not show an apparent reduction in contractile function in single cardiomyocytes superfused with buffer containing basal Ca²⁺ concentration (1.2 mM). In normal cardiomyocytes, increased Ca²⁺ concentration (2.5 mM) enhanced cardiomyocyte contraction (FIG. 6C-a). However, increased Ca²⁺ concentration failed to increase contraction of MLCK knocked-down cardiomyocytes (FIG. 6C-b, D). MLCK knocked-down cardiomyocytes may have a limited contractile reserve in response to increased demands. If the phenotype observed in a single cardiomyocyte is translated to the whole heart, the heart with reduced expression of cardiac MLCK would not be able to maintain proper contractile function under increased demand, and would be susceptible to heart failure. Indirect evidence supports that the level of cardiac MLCK protein expression is decreased in mice with severe heart failure (FIG. 7), that MLC2v phosphorylation is decreased in patients with heart failure, and that the expression of nonphosphorylatable MLC2v (Ser14, 15 and 19 to Ala mutations) in transgenic mouse hearts leads to heart failure (35-37).

Although MLCK was knocked-down successfully using adenoviral-mediated RNAi (see FIG. 5C) MLC2 phosphorylation levels were reduced by 55%. This result, combined with relatively high expression level of cardiac MLCK in the heart, suggests that cardiac MLCK is a new key MLC2 kinase in cardiomyocytes. However a significant amount of MLC2v was still phosphorylated in cardiac MLCK-knockdown cardiomyocytes. Similar results were also observed in skeletal muscle purified from homozygous knockout mice for skeletal. MLCK (15). In our study, this could be due to a low remaining level of cardiac MLCK in cardiomyocytes and/or contribution by other MLC2 kinases, including smooth muscle-MLCK (6) and PKC (38) which are also expressed in cardiomyocytes.

Cardiac MLCK has a similar overall structure to the known skeletal and smooth muscle MLCKs, including a conserved catalytic- and adjacent putative Ca²⁺/calmodulin binding domains at the carboxyl-terminus. However, its catalytic activity was not observed to be regulated by Ca⁺/calmodulin or EGTA (5 mM) in our vitro kinase assay. It is possible that, in cardiomyocytes, cardiac MLCK forms a protein complex with other factors which may regulate its catalytic activity Ca²⁺/calmodulin-dependently. It is well known that under physiological conditions, the level of MLC2v phosphorylation in the myocardium is maintained relatively constant by well-balanced MLCK induced phosphorylation and phosphatase-induced dephosphorylation (31). In addition, elevation of cytoplasmic Ca²⁺ induced by infusion of Ca²⁺ or positive inotropic agents did not consistently increase MLC2v phosphorylation (39-41). These previous observations may be explained by Ca²⁺/calmodulin-independent cardiac MLCK's activity which has a high Km, but relatively low Km. Perhaps this could reflect a functional requirement for this kinase in the heart or cardiomyocyte that contracts and relaxes constantly.

The amino-terminus of cardiac MLCK has no homologies to known proteins, suggesting that the unique amino-terminus may have additional functions, including regulation of kinase activity, localization of proteins, or interaction with other proteins. Cardiac MLCK was predominantly distributed diffusely throughout the cytoplasm. Occasionally, MLCK showed a striated expression pattern but was not overlapping with MLC2v in A bands, instead it was overlapping with actin in I bands. This finding may be interpreted that the interaction between MLCK and its substrate, MLC2v may be transient. It is also possible that cardiac MLCK may have additional functions including phosphorylation of other proteins. Previous studies demonstrated that intracellular localization of smooth muscle MLCK (long-form) depends on the actin-binding sequence consisting of repeat motifs (DFRXXL) located in the amino terminus (32,42). Although cardiac MLCK and actin are occasionally co-localized, cardiac MLCK does not have conservation in the actin-binding sequences. Thus, different domains in cardiac MLCK may regulate its intracellular localization.

Previous studies demonstrated that phosphorylation of MLCKs appears to be important for regulating MLCK activities. With smooth muscle MLCK, the most well-studied effect of MLCK phosphorylation occurs upon phosphorylation of one of two serine residues in the carboxyl-terminus of the Ca²⁺/calmodulin-binding sequence by protein kinase A, protein kinase C, CaMKII and PAK (3). Although cardiac MLCK is phosphorylated at serine residue(s), these two serine residues are not conserved in cardiac MLCK (789R, 790K in FIG. 2B). In addition, autophosphorylation has been reported for regulation or modulation of MLCK catalytic activity in skeletal-MLCK (amino-terminus to the catalytic domain), smooth muscle-MLCK (in the calmodulin binding domain and carboxyl-terminus to this domain) and Dictyostelium MLCK (between the catalytic and calmodulin binding domain) (43-46). Our in vitro kinase assay suggests potential autophosphorylation of cardiac MLCK; however, none of the reported serine-threonine residues are conserved in cardiac MLCK. Based on the amino acid sequence, cardiac MLCK most likely has different signaling pathways and self-regulatory mechanisms to regulate its function, which will be examined in future experiments.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Alberts, B., Johnson, A., Lewis, J., Raff, M., Roberts, K., and Walter, P. (2002) Molecular biology of the cell, 4 th. Ed., Garland Publishing, Inc., New York, N.Y.
2. Murthy, K. S. (2006) Annu Rev Physiol 68, 345-374
3. Kamm, K. E., and Stull, J. T. (2001) J Biol Chem 276, 4527-4530
4. Stelzer, J. E., Patel, J. R., and Moss, R. L. (2006) J Gen Physiol 128, 261-272
5. Moss, R. L., and Fitzsimons, D. P. (2006) Circ Res 99, 225-227
6. Herring, B. P., Dixon, S., and Gallagher, P. J. (2000) Am J Physiol Cell Physiol 279, C1656-1664
7. Blue, E. K., Goeckeler, Z. M., Jin, Y., Hou, L., Dixon, S. A., Herring, B. P., Wysolmerski, R. B., and Gallagher, P. J. (2002) Am J Physiol Cell Physiol 282, C451-460
8. Kemp, B. E., Pearson, R. B., and House, C. (1983) Proc Natl Acad Sci USA 80, 7471-7475
9. Herring, B. P., Gallagher, P. J., and Stull, J. T. (1992) J Biol Chem 267, 25945-25950
10. Ikebe, M., Reardon, S., Schwonek, J. P., Sanders, C. R., 2nd, and Ikebe, R. (1994) J Biol Chem 269, 28165-28172
11. Nunnally, M. H., Rybicki, S. B., and Stull, J. T. (1985) J Biol Chem 260, 1020-1026
12. Aoki, H., Sadoshima, J., and Izumo, S. (2000) Nat Med 6, 183-188
13. Davis, J. S., Hassanzadeh, S., Winitsky, S., Lin, H., Satorius, C., Vemuri, R., Aletras, A. H., Wen, H., and Epstein, N. D. (2001) Cell 107, 631-641
14. Herring, B. P., Stull, J. T., and Gallagher, P. J. (1990) J Biol Chem 265, 1724-1730
15. Zhi, G., Ryder, J. W., Huang, J., Ding, P., Chen, Y., Zhao, Y., Kamm, K. E., and Stull, J. T. (2005) Proc Natl Acad Sci USA 102, 17519-17524
16. Ohlmann, P., Tesse, A., Loichot, C., Ralay Ranaivo, H., Roul, G., Philippe, C., Watterson, D. M., Haiech, J., and Andriantsitohaina, R. (2005) Am J Physiol Heart Circ Physiol 289, H2342-2349
17. Mayans, O., van der Ven, P. F., Wilm, M., Mues, A., Young, P., Furst, D. O, Wilmanns, M., and Gautel, M. (1998) Nature 395, 863-869
18. Hardy, S., Kitamura, M., Harris-Stansil, T., Dai, Y., and Phipps, M. L. (1997) J Virol 71, 1842-1849
19. Kasahara, H., and Aoki, H. (2005) Methods Mol Med 112, 155-172
20. Lennon, G., Auffray, C., Polymeropoulos, M., and Soares, M. B. (1996) Genomics 33, 151-152
21. Sadoshima, J., Jahn, L., Takahashi, T., Kulik, T. J., and Izumo, S. (1992) J Biol Chem 267, 10551-10560
22. Kagaya, Y., Weinberg, E. O., Ito, N., Mochizuki, T., Barry, W. H., and Lorell, B. H. (1995) J Clin Invest 95, 2766-2776
23. Kuznetsov, V., Pak, E., Robinson, R. B., and Steinberg, S. F. (1995) Circ Res 76, 40-52
24. Pashmforoush, M., Lu, J. T., Chen, H., Amand, T. S., Kondo, R., Pradervand, S., Evans, S. M., Clark, B., Feramisco, J. R., Giles, W., Ho, S. Y., Benson, D. W., Silberbach, M., Shou, W., and Chien, K. R. (2004) Cell 117, 373-386
25. Hayashi, S., Lewis, P., Pevny, L., and McMahon, A. P. (2002) Mech Dev 119 Suppl 1, S97-S101
26. Kasahara, H., Wakimoto, H., Liu, M., Maguire, C. T., Converso, K. L., Shioi, T., Huang, W. Y., Manning, W. J., Paul, D., Lawitts, J., Berul, C. I., and Izumo, S. (2001) Clin Invest 108, 189-201
27. Kasahara, H., and Izumo, S. (1999) Mol Cell Biol 19, 526-536
28. Lints, T. J., Parsons, L. M., Hartley, L., Lyons, I., and Harvey, R. P. (1993) Development 119, 419-431
29. Komuro, I., and Izumo, S. (1993) Proc Natl Acad Sci USA 90, 8145-8149
30. Kasahara, H., Bartunkova, S., Schinke, M., Tanaka, M., and Izumo, S. (1998) Circ Res 82, 936-946
31. Olsson, M. C., Patel, J. R., Fitzsimons, D. P., Walker, J. W., and Moss, R. L. (2004) Am J Physiol Heart Circ Physiol 287, H2712-2718
32. Soderling, T. R., and Stull, J. T. (2001) Chem Rev 101, 2341-2352
33. Hannenhalli, S., Putt, M. E., Gilmore, J. M., Wang, J., Parmacek, M. S., Epstein, J. A., Morrisey, E. E., Margulies, K. B., and Cappola, T. P. (2006) Circulation 114, 1269-1276
34. Sanger, J. W., Ayoob, J. C., Chowrashi, P., Zurawski, D., and Sanger, J. M. (2000) Adv Exp Med Biol 481, 89-102; discussion 103-105
35. van Der Velden, J., Klein, L. J., Zaremba, R., Boontje, N. M., Huybregts, M. A., Stooker, W., Eijsman, L., de Jong, J. W., Visser, C. A., Visser, F. C., and Stienen, G. J. (2001) Circulation 104, 1140-1146
36. Palmer, B. M. (2005) Heart Fail Rev 10, 187-197
37. Sanbe, A., Fewell, J. G., Gulick, J., Osinska, H., Lorenz, J., Hall, D. G., Murray, L. A., Kimball, T. R., Witt, S. A., and Robbins, J. (1999) J Biol Chem 274, 21085-21094
38. Venema, R. C., Raynor, R. L., Noland, T. A., Jr., and Kuo, J. F. (1993) Biochem J 294 (Pt 2), 401-406
39. Jeacocke, S. A., and England, P. J. (1980) Biochem J 188, 763-768
40. High, C. W., and Stull, J. T. (1980) Am J Physiol 239, H756-764
41. Herring, B. P., and England, P. J. (1986) Biochem J 240, 205-214
42. Poperechnaya, A., Varlamova, O., Lin, P. J., Stull, J. T., and Bresnick, A. R. (2000) J Cell Biol 151, 697-708
43. Gao, Z. H., Moomaw, C. R., Hsu, J., Slaughter, C. A., and Stull, J. T. (1992) Biochemistry 31, 6126-6133
44. Sobieszek, A. (1995) Biochemistry 34, 11855-11863
45. Tokui, T., Ando, S., and Ikebe, M. (1995) Biochemistry 34, 5173-5179
46. Tokumitsu, H., Hatano, N., Inuzuka, H., Ishikawa, Y., Uyeda, T. Q., Smith, J. L., and Kobayashi, R. (2004) J Biol Chem 279, 42-50

1. A method of modifying a level of expression of a cardiac specific myosin light chain kinase (MLCK) in a cell of a subject comprising expressing in the cell a recombinant genetic construct comprising a promoter operably linked to a nucleic acid molecule encoding a MLCK polypeptide comprising SEQ ID NO: 1 or 2.

2. A method for identifying a subject at risk of heart failure comprising detecting the level of MLCK expression in a cardiac sample of a test subject and comparing the level of MLCK expression of said test subject with the level of MLCK expression in a cardiac sample or cardiac samples of a control subject or population of control subjects, wherein a decrease in the level of MLCK expression for the test subject as compared to the control subject or population of control subjects indicates that the test subject is at risk of heart failure.

3. The method of claim 2, wherein the cardiac sample comprises cardiomyocytes.

4. The method of claim 2, wherein the cardiac sample comprises cardiac fibers.

5. A method for identifying a candidate compound that modulates the activity of the MLCK polypeptide comprising:
(a) contacting a MLCK polypeptide with a candidate compound; and
(b) determining whether the candidate compound modulates MLCK activity.

6. The method according to claim 5, wherein said MLCK polypeptide is expressed in a host cell and said host cell is contacted with said candidate compound.

7. The method according to claim 5, wherein said MLCK polypeptide is naturally expressed by a cardiomyocyte or by a sample of cardiac fibers and said cardiomyocyte is contacted with said candidate compound.

8. The method according to claim 5, wherein the ability of the candidate compound to modulate MLCK activity is determined by a kinase assay.

9. A method for identifying a candidate compound that modulates the expression of the MLCK polypeptide comprising:
(a) contacting a cell expressing a MLCK polypeptide with a candidate compound; and
(b) determining whether the candidate compound modulates MLCK polypeptide expression.

10. The method according to claim 9, wherein said cell is a host cell comprising a polynucleotide encoding an MLCK polypeptide.

11. The method according to claim 9, wherein said cell is a cardiomyocyte or sample of cardiac fibers.

12. The method according to claim 9, wherein the effect of said candidate compound on MLCK expression is determined by:
a) detecting the level of MLCK mRNA expression in a cell or population of cells contacted with said candidate compound;
b) detecting the level of MLCK mRNA expression in a cell or population of cells that have not been contacted with said candidate compound; and
c) comparing the levels of MLCK mRNA expression for the cells of (a) and (b) to identify compounds that modulate MLCK mRNA expression.

13. The method according to claim 9, wherein the effect of said candidate compound on MLCK expression is determined by:
a) detecting the level of MLCK polypeptide expression in a cell or population of cells contacted with said candidate compound;
b) detecting the level of MLCK polypeptide expression in a cell or population of cells that have not been contacted with said candidate compound; and
c) comparing the levels of MLCK polypeptide expression for the cells of (a) and (b) to identify compounds that modulate the expression of MLCK polypeptide.

14. The method according to claim 13, wherein the level of MLCK expression is determined by a kinase assay.

15. The method according to claim 13, wherein the level of MLCK expression is determined by an immunoassay to quantify the amount of MLCK polypeptide expressed by the cells of (a) and/or (b).

16. The method according to claim 13, wherein the level of MLCK polypeptide expressed by cells contacted with said candidate compound is compared to the levels of MLCK expression for cells not contacted with a candidate compound in a previous test.

17. The method according to claim 14, wherein the level of MLCK polypeptide expressed by cells contacted with said candidate compound is compared to the levels of MLCK expression for cells not contacted with a candidate compound in a previous test.

18. The method according to claim 15, wherein the level of MLCK polypeptide expressed by cells contacted with said candidate compound is compared to the levels of MLCK expression for cells not contacted with a candidate compound in a previous test.

19. The method according to claim 13, wherein the level of MLCK expression is determined by a nucleic acid assay that quantifies the amount of MLCK polypeptide expressed by the control cells or the cells contacted with a candidate compound.

20. The method according to claim 15, wherein the level of MLCK expression is determined by a nucleic acid assay that quantifies the amount of MLCK polypeptide expressed by the control cells or the cells contacted with a candidate compound.

21. The method according to claim 16, wherein the level of MLCK expression is determined by a nucleic acid assay that quantifies the amount of MLCK polypeptide expressed by the control cells or the cells contacted with a candidate compound.

22. The method according to claim 17, wherein the level of MLCK expression is determined by a nucleic acid assay that quantifies the amount of MLCK polypeptide expressed by the control cells or the cells contacted with a candidate compound.

23. The method according to claim 18, wherein the level of MLCK expression is determined by a nucleic acid assay that quantifies the amount of MLCK polypeptide expressed by the control cells or the cells contacted with a candidate compound.

24. The method according to claim 19, wherein said nucleic acid assay is a quantitative PCR or quantitative real time PCR assay.

25. The method according to claim 20, wherein said nucleic acid assay is a quantitative PCR or quantitative real time PCR assay.

26. The method according to claim 21, wherein said nucleic acid assay is a quantitative PCR or quantitative real time PCR assay.

27. The method according to claim 22, wherein said nucleic acid assay is a quantitative PCR or quantitative real time PCR assay.

28. The method according to claim 23, wherein said nucleic acid assay is a quantitative PCR or quantitative real time PCR assay.
